(19) United States
(12) Patent Application Publication
(10) Pub. No.: US 2022/0054001 A1
(43) Pub. Date: Feb. 24, 2022

(54) DEVICES, SYSTEMS AND METHODS FOR DIAGNOSIS AND ASSESSMENT OF RECTAL CANCER TREATMENT RESPONSE

(71) Applicant: Washington University, St. Louis, MO (US)

(72) Inventors: Quing Zhu, St. Louis, MO (US); Shihab Uddin, St. Louis, MO (US); William Chapman, St. Louis, MO (US); Xiandong Leng, St. Louis, MO (US); Matthew Mutch, St. Louis, MO (US)

(73) Assignee: Washington University, St. Louis, MO (US)

(21) Appl. No.: 17/408,371

(22) Filed: Aug. 20, 2021

Related U.S. Application Data
(60) Provisional application No. 63/067,953, filed on Aug. 20, 2020.

(51) Int. Cl. A61B 1/31; A61B 1/07; A61B 5/00; G06N 3/04; G06T 7/37

(52) U.S. Cl. CPC A61B 1/31; A61B 1/07; A61B 5/7275; A61B 5/0004; G06T 2207/10132; G06N 3/04; G06T 7/37; G06T 2207/20084; G06T 2207/30096; A61B 5/742

(57) ABSTRACT

A system for determining a probability of normal rectal tissue composition within a region of interest of an ultrasound or photoacoustic image of the rectal tissue is disclosed. The system includes a computing device with at least one processor configured to receive at least one of a photoacoustic image and an ultrasound image; select a region of interest within the at least one of a photoacoustic image and an ultrasound image; transform the region of interest into the probability of normal rectal tissue composition using a CNN model; and display the probability of normal rectal tissue composition to an operator of the system.

### CROSS-REFERENCE TO RELATED APPLICATIONS

This application claims priority from U.S. Provisional Application Ser. No. 63/067,953 filed on Aug. 20, 2020, which is incorporated herein by reference in its entirety.

### STATEMENT REGARDING FEDERALLY SPONSORED RESEARCH OR DEVELOPMENT

This invention was made with government support under CA151570, CA228047, CA237664, and CA009621 awarded by the National Institutes of Health. The government has certain rights in the invention.

### SUMMARY

The present disclosure generally relates to devices, systems, and methods of diagnosing rectal cancers and assessing the response to treatments.

In one aspect, a system for determining a probability of normal rectal tissue composition within a region of interest of an ultrasound or photoacoustic image of the rectal tissue is disclosed. The system includes a computing device with at least one processor and a non-volatile computer-readable memory, the non-volatile computer-readable memory containing a plurality of instructions executable on the at least one processor, the plurality of instructions comprising a CNN component configured to: receive at least one of a photoacoustic image and an ultrasound image; select a region of interest within the at least one of a photoacoustic image and an ultrasound image; transform the region of interest into the probability of normal rectal tissue composition using a CNN model; and display the probability of normal rectal tissue composition to an operator of the system.

In another aspect, an endorectal imaging probe for obtaining co-registered ultrasound and photoacoustic images of a rectal tissue of a subject is disclosed. The probe includes a handle comprising an integrated stepper motor and a light source; a hollow shaft containing a hollow axle, the hollow axle coupled to the stepper motor at a proximal end; an imaging head coupled to a distal end of the hollow axle, the imaging head comprising: a toroidal ultrasonic transducer mounted to an outer surface of the imaging head to detect acoustic signals produced outside of the imaging head, the toroidal ultrasonic transducer comprising a center hole aligned perpendicularly to the longitudinal axis of the probe, the toroidal ultrasonic transducer operatively connected to a remote pulser/receiver device via an ultrasonic transducer cable extending distally through the hollow axle; an optical fiber coupled to a light source at a proximal end and extending distally through the hollow axle to a distal fiber end positioned within the imaging head; and a prism positioned within the imaging head to direct light delivered through the optic fiber to a segment of multimode optical fiber positioned within the center hole of the transducer, the segment of multimode optical fiber configured to direct light perpendicularly outward from the imaging head.

### BACKGROUND OF THE DISCLOSURE

Colorectal cancer is the third most common cancer diagnosed in both men and women in the United States as well as in Veteran Affair (VA) hospitals. Rectal cancer is a prevalent disease that requires complex, coordinated care to achieve maximal survival. Multiple trials evaluating various modes of incorporating both chemotherapy and chemoradiation treatment in the neoadjuvant (preoperative) setting, referred to as “total neoadjuvant therapy (TNT), have reported optimistic results. TNT offers a chance for early delivery of aggressive systemic treatment against the development and progression of micrometastases, potentially increasing survival rates in locally advanced rectal cancer (LARC). Additionally, pathological complete response (pCR, no residual cancers) rates increased significantly with the administration of neoadjuvant chemotherapy (NAC). The pCR rate was associated with a significantly lower local recurrence, 5-year distant relapse, and significantly improved 5-year disease-free survival and 5-year overall survival. Furthermore, TNT provides the opportunity to assess an individual patient's chemosensitivity and tumor response prior to surgery. This can lead to better risk stratification and identification of patients who may not require surgery or need more therapy. In recent years, ground-breaking clinical studies have explored a nonoperative strategy—called “watch and wait”—that allows patients who have achieved complete tumor destruction (pCR) with radiation and chemotherapy to avoid surgical resection altogether with good long-term oncologic and functional outcomes. There is tremendous interest and desire for organ preservation in rectal cancer, partly driven by patients who want to preserve a good quality of life.

However, an important concern arising from the “watch and wait” approach is how to accurately access tumor regression and confidently identify patients with pCR. Biopsy has been associated with an 11% negative-predictive value, and reliance on endoscopy is limited by reports of persistent mucosal ulceration in 66% of patients with pCR. Current management of rectal malignancies relies on detailed radiographic testing for both staging and treatment response evaluations. MRI has become the critical staging tool for newly diagnosed rectal cancers. However, monitoring tumors after chemotherapy and radiation with MRI is much more difficult because post-treatment imaging is confounded by fibrotic reaction and edema, thus making it extremely difficult to identify complete or near-complete responders from those with surviving malignant tissue. Functional MRI, in particular diffusion-weighted imaging (DWI), improves the ability of MRI in assessment of post-treatment response, however, the resolution and accuracy are still problematic for clinical use. Currently, endoscopic ultrasound (EUS) is recommended as a second line modality for rectal cancer staging after initial diagnosis in cases where MRI is contraindicated. However, EUS has low sensitivity in estimating response after neoadjuvant treatment before surgery, due to peritumoral inflammation, edema, necrosis, and fibrosis of the neoplastic tissue. Doppler US, useful for estimating the presence, the density, or absence of vascular signals in the large blood vessels, is not sensitive enough to detect slow and low-volume flow in smaller vessels of gastrointestinal organs. Newer US technologies include contrast-enhanced US, which uses microbubbles to study tumor angiogenesis (CEUS), and US elastography for evaluating tumor stiffness. Currently, there is limited data on using CEUS to study rectal cancer perfusion. No data are available on using any of these technologies to evaluate a treated rectal tumor bed and assess treatment response. 18F-FDG PET/CT has a limited role in measuring post-treatment response. The identification of molecular biomarkers to predict treatment response has been of great interest. However, to date none has currently reached the clinic. Clearly, there is an urgent need for additional imaging modalities to improve the current standard of care (SOC) and better predict which patients have achieved pCR and therefore can safely undergo a “watch-and-wait” approach.

In the past decade, with advances in lasers, ultrasound transducers, and tomographic reconstruction techniques, photoacoustic imaging (PAI) has seen immense growth, providing unprecedented spatial resolution and functional information at depths ranging from several millimeters up to several centimeters. PAI is a hybrid imaging technology that uses a short-pulsed laser to excite tissue. The resulting acoustic (or photoacoustic) waves are generated from thermoelastic expansion due to transient temperature rises. They are then acquired by US transducers and used to image the optical absorption distribution, which in turn reveals optical contrast. Optical contrast is directly related to microvessel networks and thus to tumor angiogenesis, a key process for tumor growth and metastasis.

Laser delivery methods play an important role in probe design for high imaging quality and deep tissue penetration. The development of laser and ultrasound detection has enabled the development of in vivo transvaginal imaging using co-registered ultrasound and photoacoustic tomography (PAT). To deliver light into the PAT imaging probe, the multimode fiber is a typical choice. However, the options of fiber diameter and numerical aperture (NA) are limited, leading to a small divergence angle and a high light fluence hotspot on the tissue surface. If the fiber is directly in contact with tissue, the light fluence can easily exceed the maximum permissible exposure (MPE) (˜28 mJ/cm² @ 780 nm). Although various methods to reduce the fluence and hotspot at skin surface have been proposed, energy loss and manufacturing difficulty still pose significant challenges to the delivery of light at a fluence suitable for photoacoustic tomography (PAT).

The inclusion of a diffuser within the delivery optics of a laser system is one general method of reducing fluence of light delivered to a tissue surface. Although some existing diffuser designs for optic fiber tips are relatively compact, the performance achieved to date is more suitable for endoscope optical imaging, rather than photoacoustic imaging. One existing fiber diffuser tip design includes a fiber tip with a conical air pocket formed by fusing a section of hollow optical fiber using an electric arc-discharge process. A laser beam injected to the air-pocket fiber interface undergoes total internal reflection and changes direction to generate a larger laser spot and/or to generate side illumination. In another existing design, the fiber tip includes a taper region used to leak light, as well as a silica or sapphire tip region full of air bubbles or other light scattering materials to scatter the light, thereby reducing the tissue surface fluence. In an additional existing design, cracks formed on the fiber tip generate reflections and refractions, thus diffusing light. Another additional existing design, a bullet-shaped fiber tip deflects and refracts light, but this design is relatively difficult to fabricate and the scattering effect is difficult to control.

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1A is an image and schematic illustration of a fiber tip diffuser.

FIG. 1B is an image showing a fluence pattern of a fiber tip output with and without a fiber tip diffuser.

FIG. 2A contains a set of color maps summarizing fiber tip diffuser output patterns from simulations of: normal fiber tip (upper-left), soda-lime glass (n=1.52) (upper-right), PMMA (n=1.49) (lower-left), and silica (n=1.458) (lower-right).

FIG. 2B is a graph comparing fluence and energy loss of different fiber tip diffusers containing different microsphere materials and sizes.

FIG. 3A is a graph summarizing fluence distribution and MPE input energy for fiber tip diffusers with different microsphere concentrations.

FIG. 3B is a graph summarizing simulated and empirically measured fluence distributions in a calibrated intralipid solution at different depths.

FIG. 4A contains a series of color maps summarizing the simulated fluence distribution of a four-fiber transvaginal probe output fluence at various depths within a calibrated intralipid solution, in which each fiber ends in a normal (non-diffuser) tip.

FIG. 4B contains a series of color maps summarizing the simulated fluence distribution of a four-fiber transvaginal probe output fluence at various depths within a calibrated intralipid solution, in which each fiber ends in a 20:10 fiber tip diffuser.

FIG. 5A is a graph summarizing simulated fluence as a function of depth within a calibrated intralipid solution for four-fiber transvaginal probes with normal (non-diffuser) and 20:10 fiber tip diffuser tips for various input laser energies.

FIG. 5B is a graph FIG. 5A is a graph summarizing empirically measured fluence as a function of depth within a calibrated intralipid solution for four-fiber transvaginal probes with normal (non-diffuser) and 20:10 fiber tip diffuser tips for various input laser energies.

FIG. 6 is a block diagram schematically illustrating a system in accordance with one aspect of the disclosure.

FIG. 7 is a block diagram schematically illustrating a computing device in accordance with one aspect of the disclosure.

FIG. 8 is a block diagram schematically illustrating a remote or user computing device in accordance with one aspect of the disclosure.

FIG. 9 is a block diagram schematically illustrating a server system in accordance with one aspect of the disclosure.

FIG. 10A is an image of a PAM endoscope in accordance with one aspect of the disclosure.

FIG. 10B is an image of the scales on a water channel of the PAM endoscope of FIG. 10A.

FIG. 10C is an image of the endoscope of FIG. 10A mounted in a proctoscope with a balloon on the tip of the imaging head.

FIG. 11 is a schematic diagram showing the architecture of a CNN model in accordance with one aspect of the disclosure.

FIG. 12A contains co-registered PAM and US images showing ROIs of residual cancer tissue; ROIs are denoted by green dashed line boxes. PAM ROIs are cropped from PAM images, and US ROIs are cropped from US images.

FIG. 12B contains co-registered PAM and US images showing ROIs of normal tissue, area in blue boxes; ROIs are denoted by blue dashed line boxes. PAM ROIs are cropped from PAM images, and US ROIs are cropped from US images.

FIG. 13A is a histogram summarizing the first order statistical features calculated from malignant rectal tissue PAM ROIs.

FIG. 13B is a histogram summarizing the first order statistical features calculated normal rectal tissue PAM ROIs.

FIG. 14A is a histogram summarizing the first order statistical features calculated from malignant rectal tissue ultrasound (US) ROIs.

FIG. 14B is a histogram summarizing the first order statistical features calculated normal rectal tissue US ROIs.

FIG. 15A is a graph summarizing the average ROC of a CNN training data set for different combinations of features set. The features were extracted from PAM images. The 95% CIs are indicated in parentheses.

FIG. 15B is a graph summarizing the average ROC of a CNN testing data set for different combinations of features set. The features were extracted from PAM images. The 95% CIs are indicated in parentheses.

FIG. 16A is a graph summarizing the average ROC of a CNN training data set for different combinations of features set. The features were extracted from US images. The 95% CIs are indicated in parentheses.

FIG. 16B is a graph summarizing the average ROC of a CNN testing data set for different combinations of features set. The features were extracted from US images. The 95% CIs are indicated in parentheses.

FIG. 17A is a graph summarizing the average ROC of PAM-CNN model training and validation. The 95% CIs are indicated in parentheses.

FIG. 17B is a graph summarizing the average ROC of PAM-CNN model testing. The 95% CIs are indicated in parentheses.

FIG. 18A is a graph summarizing the average ROC of US-CNN model training and validation. The 95% CIs are indicated in parentheses.

FIG. 18B is a graph summarizing the average ROC of US-CNN model testing. The 95% CIs are indicated in parentheses.

FIG. 19A is a boxplot summarizing the mean histogram feature of PAM images. Each plotted point represents the histogram feature in one ROI.

FIG. 19B is a boxplot summarizing the standard deviation (STD) histogram feature of PAM images. Each plotted point represents the histogram feature in one ROI.

FIG. 19C is a boxplot summarizing the skewness histogram feature of PAM images. Each plotted point represents the histogram feature in one ROI.

FIG. 19D is a boxplot summarizing the kurtosis histogram feature of PAM images. Each plotted point represents the histogram feature in one ROI.

FIG. 19E is a boxplot summarizing the energy histogram feature of PAM images. Each plotted point represents the histogram feature in one ROI.

FIG. 20A is a boxplot summarizing the mean histogram feature of US images. Each plotted point represents the histogram feature in one ROI.

FIG. 20B is a boxplot summarizing the standard deviation (STD) histogram feature of US images. Each plotted point represents the histogram feature in one ROI.

FIG. 20C is a boxplot summarizing the skewness histogram feature of US images. Each plotted point represents the histogram feature in one ROI.

FIG. 20D is a boxplot summarizing the kurtosis histogram feature of US images. Each plotted point represents the histogram feature in one ROI.

FIG. 20E is a boxplot summarizing the energy histogram feature of US images. Each plotted point represents the histogram feature in one ROI.

FIG. 21A is a drawing showing a PAM imaging head.

FIG. 21B is a drawing showing the PAM imaging head of FIG. 20A implementing a forward mode of PAM imaging; the forward view facilitates bench-top scanning.

FIG. 21C is a drawing showing the PAM imaging head of FIG. 20A implementing a side view mode of PAM imaging; the side view mode is suitable for endoscopic patient studies.

FIG. 22 is a drawing showing the light path of an endorectal probe in accordance with one aspect of the disclosure.

FIG. 23A is a photograph of a normal colon.

FIG. 23B is an ultrasound image of the normal colon tissue of FIG. 23A.

FIG. 23C is a co-registered PAM/US image of the same cross section of FIG. 23B, where white arrows refer to blood vessels in mucosa and yellow arrows refer to blood vessels in the submucosa.

FIG. 23D is an H&E histological section of normal colon tissue where yellow arrows identify blood vessels.

FIG. 23E is a photograph of an area of a colon containing colon cancer treated with chemotherapy.

FIG. 23F is an ultrasound image of the lesion of FIG. 23E; ultrasound gel fills the ulcer cavity (dashed line) but is not within the tissue.

FIG. 23G is a co-registered AR-PAM/US image of the same cross section of FIG. 23F, where white arrows refer to blood vessels in mucosa and yellow arrows refer to blood vessels in the submucosa; increased photoacoustic signal is visible around the ulcerated area.

FIG. 23H is an H&E histological section of a tumor bed within the colon tissue; residual islands of cancer cells (blue arrows) are visible several millimeters beneath the surface of the specimen.

FIG. 23I is a photograph of an area of a colon containing rectal cancer treated with chemoradiation prior to surgical resection.

FIG. 23J is an ultrasound image of the post-treated rectum tumor of FIG. 23I.

FIG. 23K is a co-registered AR-PAM/US image of the same cross section of FIG. 23J.

FIG. 23L is an H&E histological section of a tumor bed within the colon tissue where yellow arrows identify blood vessels; no residual tumor is visible.

FIG. 24A is a T2-weighted MRI showing anterior hyperintensity of residual cancer of a 68-year old patient treated with chemoradiation and imaged before surgery.

FIG. 24B is an US image from the patient of FIG. 24A; large white box marks the tumor bed and ROIs used for labeling the tumors for CNN training are marked by smaller white boxes inside tumor bed.

FIG. 24C is a co-registered PAM/US image of the same cross section of FIG. 24B; large white box marks the tumor bed and ROIs used for labeling the tumors for CNN training are marked by smaller white boxes inside tumor bed.

FIG. 24D is an H&E histological section of a colon tumor tissue border.

FIG. 24E is an US image of normal colon tissue; ROIs used for CNN training are marked by smaller white boxes.

FIG. 24F is a co-registered PAM/US image of the same cross section of FIG. 24E; ROIs used for CNN training are marked by smaller white boxes.

FIG. 24G is an H&E histological section of a normal colon tissue border showing rich blood vessels in mucosa and submucosa.

FIG. 25 is a schematic diagram showing the architecture of a CNN model for identifying normal colorectal tissue in accordance with one aspect of the disclosure.

FIG. 26A contains a T2-weighted MR image (left) and a diffusion-weighted MRI image (right) of colon tissue treated with preoperative radiation and chemotherapy.

FIG. 26B is an endoscopic image of the colon tissue of FIG. 26A.

FIG. 26C is an endorectal US image of the colon tissue of FIG. 26A.

FIG. 26D is a co-registered PAM/US image of the colon tissue of FIG. 26A.

FIG. 26E is an H&E histological section of the colon tissue of FIG. 26A.

FIG. 26F is an endorectal US image of healthy colon tissue distal to the tissue of FIG. 26A.

FIG. 26G is a co-registered PAM/US image of healthy colon tissue distal to the tissue of FIG. 26A.

FIG. 26H is an H&E histological section of healthy colon tissue distal to the tissue of FIG. 26A.

FIG. 27A is a graph summarizing ROC of a PAM-CNN model tested on patients.

FIG. 27B is a graph summarizing ROC of a US-CNN model tested on patients.

FIG. 28 is a co-registered PAM/US image of a colon overlaid with 8 sectors (blue lines) used for initialization of automated scanning and diagnosis; manually selected ROIs are denoted by overlaid blue rectangles. Sectors are identified using an ellipsoid fitting and segmentation code.

FIG. 29A is a co-registered PAM-velocity map/US image of tumor-containing colon tissue.

FIG. 29B is a co-registered PAM-velocity map/US image of healthy colon tissue.

FIG. 30A is a graph summarizing area under ROC at different training set sizes for a PAM-CNN model.

FIG. 30B is a graph summarizing area under ROC at different training set sizes for an US-CNN model.

FIG. 31 is a schematic diagram sowing the elements of an AR-PAE system in accordance with one aspect of the disclosure.

FIG. 32A is a co-registered PAM-velocity map/US image of cancerous colon tissue.

FIG. 32B is the US portion of the image of FIG. 32A with overlaid boxes denoting ROIs.

FIG. 32C is the PAM portion of the image of FIG. 32A with overlaid boxes denoting ROIs.

FIG. 32D is a co-registered PAM-velocity map/US image of normal colon tissue.

FIG. 32E is the US portion of the image of FIG. 32E with overlaid boxes denoting ROIs.

FIG. 32F is the PAM portion of the image of FIG. 32E with overlaid boxes denoting ROIs.

FIG. 33A contains T2-weighted MRI (left) and diffusion weighted images (DWI, right) of colon tissues of a first patient with residual cancer, as denoted by overlaid green arrows. The images show intermediate T2 signal and DWI hyperintensity corresponding to diffusion restriction in residual cancer.

FIG. 33B is US image of the rectum of the patient of FIG. 33A with the tumor denoted by a dashed line box.

FIG. 33C is a co-registered US and PA image of the same tissue shown in FIG. 33B.

FIG. 33D is an H&E image of the lesion area, showing a few tiny vessels at the surface (arrows), while a majority of the lesion region lacks blood vessels.

FIG. 33E contains a T2-weighted MRI (left) and diffusion weighted images (DWI, right) of colon tissues of a second patient with residual cancer, as denoted by overlaid green arrows. The images show intermediate to low T2 signal and diffusion restriction from 10 o'clock to 6 o'clock, as indicated by green arrows.

FIG. 33F is an US image of the rectum of the patient of FIG. 33E with the tumor denoted by a dashed line box.

FIG. 33G is a co-registered US and PA image of the same tissue shown in FIG. 33F.

FIG. 33H is an H&E image of the lesion area from the patient of FIG. 33E, showing the interface of the normal and cancer regions.

FIG. 33I contains a T2-weighted MRI (left) and diffusion weighted images (DWI, right) of colon tissues of a patient taken 3 months before surgery, showing good response to chemotherapy and radiation treatments with no residual abnormal T2 or DWI signal to indicate residual viable tumor.

FIG. 33J is an US image including the cancer region of the patient of FIG. 33I with the tumor denoted by a dashed line box.

FIG. 33K is a co-registered US and PA image of the same tissue shown in FIG. 33J.

FIG. 33L is an H&E image taken at approximately the middle of the tumor from the patient of FIG. 33I, showing that most of the area lacks blood vessels.

FIG. 33M contains a T2-weighted MRI (left) and diffusion weighted images (DWI, right) of colon tissues of a patient taken after chemotherapy and radiation treatment showing low T2 signal in the right rectal wall corresponding with fibrosis after treatment and no DWI hyperintensity to suggest a recurrent tumor.

FIG. 33N is an US image of the patient of FIG. 33M showing some acoustic attenuation contrast, as indicated by a white dashed line box.

FIG. 33O is a co-registered US and PA image of the same tissue shown in FIG. 33N showing a region lacking of vasculature.

FIG. 33P is an H&E image taken at approximately the middle of the tumor from the patient of FIG. 33M, showing a recurrence tumor of approximately 1.2 cm.

FIG. 34A contains an US image of the rectum region of a first normal patient. Overlaid number is US-CNN output indicating the probability of normal tissue.

FIG. 34B is a co-registered US and PA image of the region of FIG. 34A. Overlaid number is PAE-CNN output indicating the probability of normal tissue.

FIG. 34C is an H&E image of the normal rectal tissue of FIG. 34A showing rich vessels in the submucosa (arrows).

FIG. 34D contains an US image of the rectum region of a second normal patient. Overlaid number is US-CNN output indicating the probability of normal tissue.

FIG. 34E is a co-registered US and PA image of the region of FIG. 34D. Overlaid number is PAE-CNN output indicating the probability of normal tissue.

FIG. 34F is an H&E image of the normal rectal tissue of FIG. 34D showing rich vessels in the submucosa (arrows).

FIG. 34G contains an US image of the rectum region of a third normal patient. Overlaid number is US-CNN output indicating the probability of normal tissue.

FIG. 34H is a co-registered US and PA image of the region of FIG. 34G. Overlaid number is PAE-CNN output indicating the probability of normal tissue.

FIG. 34I is an H&E image of the normal rectal tissue of FIG. 34G showing rich vessels in the submucosa (arrows).

FIG. 34J contains an US image of the rectum region of a fourth normal patient. Overlaid number is US-CNN output indicating the probability of normal tissue.

FIG. 34K is a co-registered US and PA image of the region of FIG. 34J. Overlaid number is PAE-CNN output indicating the probability of normal tissue.

FIG. 34L is an H&E image of the normal rectal tissue of FIG. 34J showing rich vessels in the submucosa (arrows).

FIG. 34M contains an US image of the rectum region of a fifth normal patient. Overlaid number is US-CNN output indicating the probability of normal tissue.

FIG. 34N is a co-registered US and PA image of the region of FIG. 34M. Overlaid number is PAE-CNN output indicating the probability of normal tissue.

FIG. 34O is an H&E image of the normal rectal tissue of FIG. 34M showing rich vessels in the submucosa (arrows).

FIG. 35A is a graph summarizing ROC of PAE-CNN patient testing results with AUC=0.976.

FIG. 35B is a graph summarizing ROC of US-CNN patient testing results with AUC=0.758.

FIG. 35C is a graph summarizing ROC of PAE-CNN of mixed ex vivo and in vivo responders and non-responders with AUC=0.952.

FIG. 35D is a graph summarizing ROC of US-CNN of mixed ex vivo and in vivo responders and non-responders with AUC=0.539.

FIG. 36A is a graph summarizing the area under receiver operating characteristic (ROC) curve vs. different training set sizes for the PAE-CNN model.

FIG. 36B is a graph summarizing the area under receiver operating characteristic (ROC) curve vs. different training set sizes for the US-CNN model.

There are shown in the drawings arrangements which are presently discussed, it being understood, however, that the present embodiments are not limited to the precise arrangements and are instrumentalities shown. While multiple embodiments are disclosed, still other embodiments of the present disclosure will become apparent to those skilled in the art from the following detailed description, which shows and describes illustrative aspects of the disclosure. As will be realized, the invention is capable of modifications in various aspects, all without departing from the spirit and scope of the present disclosure. Accordingly, the drawings and detailed description are to be regarded as illustrative in nature and not restrictive.

### DETAILED DESCRIPTION OF THE INVENTION

Conventional radiologic modalities perform poorly in the rectal cancer post-treatment management and are often unable to differentiate residual cancer from treatment scar. In various aspects, a novel imaging system that includes an endorectal probe configured to obtain photoacoustic images co-registered with ultrasound images (PAE/US) and a deep learning neural network model (PAE-CNN) configured to classify healthy versus cancerous tissues within the PA and US images obtained by the PAE/US probe to accurately assess treatment response of colorectal tissues. As described in the examples below, the PAE-CNN models were trained, validated, and tested using ex vivo colorectal tissue. In the ex vivo setting, we found significant differences in vascular tissue signaling between normal and malignant colorectal tissues. In pilot patients who were treated by radiation and chemotherapy, similar differences were detected by the proposed imaging system, which demonstrated excellent performance, as measured by the area under a receiver-operating-characteristic curve of 0.976 using PAE-CNN. The PAE/US system coupled with the deep learning PAE-CNN model as disclosed herein accurately assesses rectal cancer treatment response and optimizes post-treatment management. The disclosed PAE/US probe and PAE-CNN model are better able to select those patients who have responded to initial treatment for nonoperative management and thereby improve patient quality of life while maintaining cancer detection sensitivity.

### US-PAM System

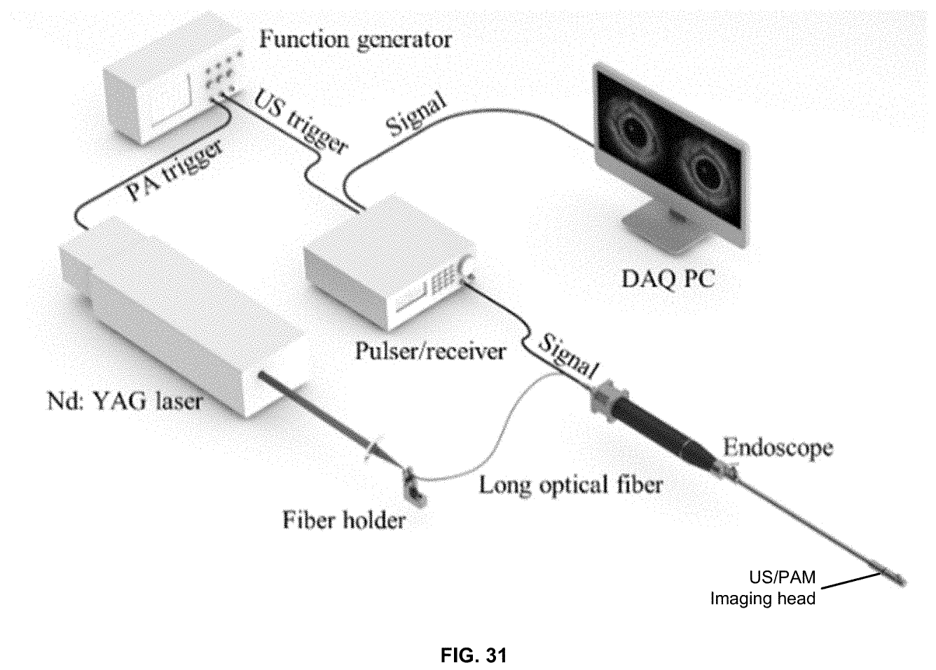

In various aspects, the acoustic resolution photoacoustic endoscopic/ultrasound (AR-PAE/US) system includes an endoscopic PAE/US imaging probe, a laser system, an ultrasound pulser/receiver, a function generator, and a data acquisition (DAQ) PC. The AR-PAE/US system is shown illustrated in FIG. 31 in one aspect. The PAE/US imaging probe, described in additional detail below, is configured to obtain co-registered photoacoustic (PA) and ultrasound (US) images of endorectal tissues via an endoscopic probe. In brief, photoacoustic images are obtained by illuminating the tissues using a series of laser pulses. In response to illumination, structures within the tissues produce photoacoustic (PA) signals in the form of ultrasound pulses that are detected by an ultrasound transducer. The PA signals are reconstructed into a PA image using any suitable reconstruction method including, but not limited to, a backprojection method. Ultrasound images are obtained by directing ultrasound pulses into the tissues using the same ultrasound transducer used to detect PA signals, followed by detection of the echoed ultrasound pulses using the same ultrasound transducer. As described in additional detail below, the use of the same ultrasound transducer in the PAE/US imaging probe to obtain the acoustic signals used to reconstruct both the PA and US images ensures that the PA and US images are co-registered.

In various aspects, the laser system of the AR-PAE/US system includes a laser configured to produce a plurality of laser pulses used to illuminate the tissues to be imaged using photoacoustic imaging. The AR-PAE/US system may include any laser source capable of producing laser pulses suitable for producing PA signals within the tissue to be imaged without limitation. In some aspects, the operational parameters of the laser may be selected to enhance an aspect of the performance of the AR-PAE/US system. By way of non-limiting example, the wavelengths of the laser pulse may be selected to transmit readily through the tissues to be imaged and/or to produce strong PA signals from structures within the tissue. In other aspects, a high pulse repetition rate may be selected to facilitate the rapid acquisition of PA signals to reduce scan time. In additional aspects, the pulse energy and pulse duration may be selected to produce robust PA signals with relatively low noise. In one exemplary aspect, the AR-PAE/US system includes an Nd:YAG laser (DPS-1064-Q, CNI Laser.com, P.R. China), operated at 1064 nm with a 1 kHz repetition rate and pulse energy of 9 mJ in 7 ns to serve as the light source, as illustrated in FIG. 31. In various aspects, the laser of the laser system is operatively coupled to the PAM/US probe by any suitable optical transmission element without limitation. Non-limiting examples of suitable optical transmission elements include free air, waveguides, and optical fibers. In one exemplary aspect, a 1.5 mm diameter BK-7 multimode fiber (MMF) couples the laser to the imaging probe.

In various aspects, AR-PAE/US system further includes a function generator configured to control the operation of the laser system and the US transducer of the imaging probe in a coordinated manner to obtain co-registered PA and US imaging data. In some aspects, the function generator is operatively coupled to the laser system and to the ultrasound transducer of the imaging probe. The function generator produces an alternating series of PA trigger signals and US trigger signals as illustrated in FIG. 31. The laser system produces laser pulses in response to each laser trigger signal as part of a PA imaging cycle and the US transducer transmits and receives US pulses in response to each US trigger signal as part of an US imaging cycle. The function generator facilitates the rapid acquisition of PA and US signals from a plurality of positions within as tissue for reconstruction as co-registered PA and US images as described herein.

In various aspects, the AR-PAE/US system further includes a pulser/receiver operatively connected to the function generator and to the US transducer of the imaging probe. The pulser/receiver is configured to operate the US transducer of the imaging probe to produce US pulses and detect echoed US pulses from the tissue to obtain US signals suitable for reconstruction into PA images of the tissue. As described above, the pulser/receiver produces transducer control signals define the operation of the US transducer during an US imaging cycle in response to US trigger signals received from the function generator.

### US-PAM Imaging Probe

In various aspects, the AR-PAE/US system further includes an US-PAM imaging head operatively coupled to the laser and an US pulser/receiver as illustrated in FIG. 31. In some aspects, the design of the US-PAM probe is compatible for use with existing medical imaging systems and devices. By way of example, the US-PAM probe may be based on an existing FDA-approved conventional endorectal ultrasound probe (BK Medical Inc. MA). With this design, the US-PAM probe fits into a standard rigid proctoscope, and is easily operated by surgeons familiar with performing standard endorectal ultrasound examinations. Making the system compatible with legacy devices may facilitate translation within the clinical community.

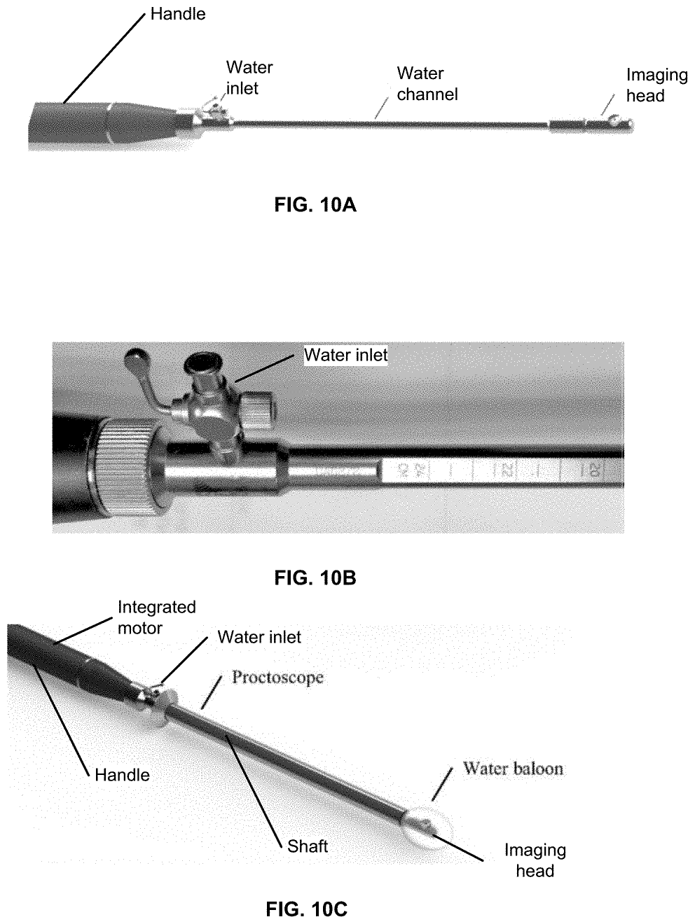

A US-AM endorectal probe is illustrated in one aspect in FIGS. 10A, 10B, and 10C. In various aspects, the endorectal probe includes a handle, a shaft, and an imaging head. A stepper motor integrated with the handle rotates the imaging head on an axle within the shaft. The axle and stepper motor are hollow, allowing the passage of the long optical fiber and ultrasonic transducer wire (see FIG. 31) from the back of the probe to the imaging head. When in use, the probe head is covered by a latex balloon which can be inflated via a water channel accessed via a water inlet positioned near the probe handle. When inflated with deionized water, the fluid within the water balloon enhances transmission of ultrasound and photoacoustic waves from the imaging head to the surrounding rectal wall.

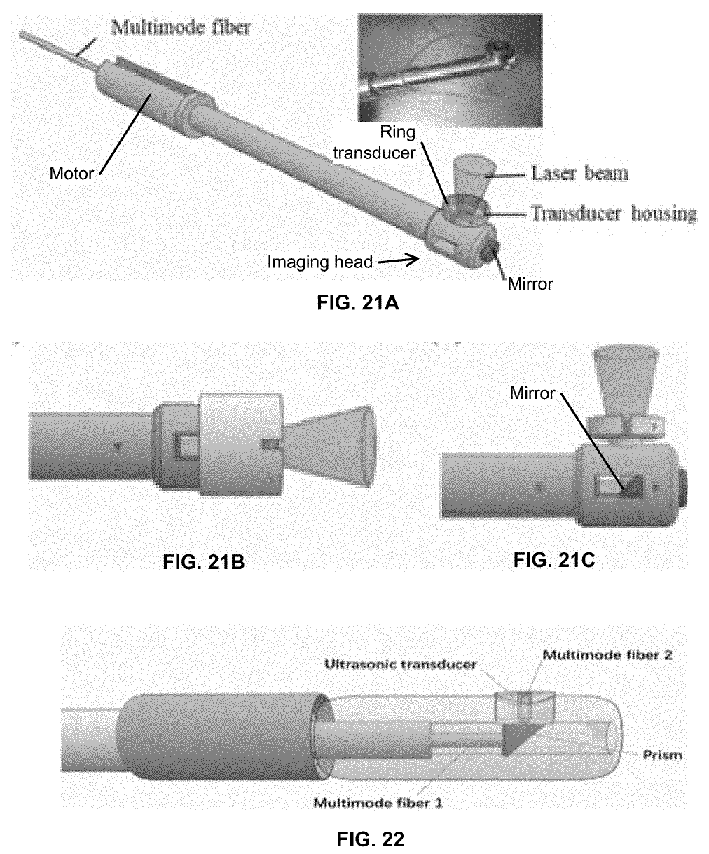

In various aspects, illustrated in FIGS. 21A, 21B, 21C, and 22, the imaging probe head contains several components that allow for side-viewing image capture over 360 degrees. In one aspect, a SF-11 prism reflects and couples the laser beam at 90° to the short 1.5 mm diameter multi-mode fiber (MMF) segment in the narrow center hole of an ultrasonic transducer (Capistrano Labs Inc, San Clemente, Calif.). The short MMF confines the laser beam, preventing it from hitting the rim of the light outlet. In another aspect, the ultrasonic transducer has an 8 mm aperture, 12.7 mm focus length, and 75% bandwidth at a 20 MHz center frequency. A function generator (FIG. 31) triggers a pulser/receiver to acquire US pulse-echo signals. Following each trigger of a US A-scan, a delayed laser trigger initiates the acquisition of a PA A-scan. In this way, the handheld AR-PAE system is able to simultaneously register US and PA data. Using a 1000 Hz laser pulse repetition rate, an imaging frame rate of one B-scan image per second has been consistently achieved. In one aspect, a 3.6 mJ laser pulse from the probe tip illuminates a tissue area of approximately 0.15 cm², resulting in a surface optical fluence of 24 mJ/cm². This energy level is well within safety standards, and the fluence is further reduced by energy diffusion caused by the latex balloon surrounding the imaging head. In another aspect, the probe tip may further include a fiber tip diffuser that reduces surface optical fluence.

In various other aspects, the PAM endoscope includes three parts: a handle, a water channel (the main body), and an imaging head, as shown in FIG. 10A. The water inlet allows water injected from a syringe to inflate a water balloon covering the imaging head to enhance ultrasound coupling. A stepper motor in the handle turns a hollow shaft in the water channel to rotate the image head 360° for full circle imaging. An optical fiber inside the hollow shaft delivers laser pulses to the imaging head. An ultrasonic transducer (20 MHz, 75% bandwidth) fixed on the imaging head both transmits and receives ultrasound signals, and also receives PA signals. An Nd:YAG laser working at 1064 nm with a 1000 Hz pulse repetition rate is the light source. A 0.15 cm2 tissue area is illuminated by 3.6 mJ laser pulses from the probe tip, resulting in a surface optical fluence of 24 mJ/cm2, which is well within the ANSI safety threshold (100 mJ/cm2) at 1064 nm. This fluence is further reduced by energy diffusion caused by the balloon.

During imaging, the PAM endoscope is inserted transanally through a proctoscope, (FIG. 10C). Ruled scales on the water channel (FIG. 10B) show how deeply the endoscope is inserted into the rectum where the images are obtained.

### Fiber Diffuser Tip

In various aspects, a novel and low-cost fiber tip diffuser using silica microspheres and UV adhesive is disclosed. The light scattering effect of this diffuser was characterized using both simulation and experiment as described above and good agreement was reached. With the disclosed diffuser, larger energy can be injected into the tissue while maintaining tissue surface laser fluence under MPE, thus enhancing the quality of PA imaging without causing tissue surface damage. The fiber tip diffuser is a useful tool for many endo-cavity photoacoustic imaging applications, such as in-vivo colorectal cancer, cervical cancer, and ovarian cancer.

In various aspects, a fiber diffuser tip is disclosed that reduces the fluence of light delivered to a tissue surface, while injecting more laser energy, thereby enhancing the photoacoustic signal generated from the tissue. Simulations and experiments have been conducted to characterize the performance of various designs of fiber diffuser tips and to assess the impact of variations in the design features on diffuser performance. In various aspects, a fiber tip diffuser to scatter light is disclosed that includes a plurality of microspheres suspended in an ultraviolet (UV) adhesive to scatter light. In various aspects, the fiber tip diffuser limits the surface fluence levels of light directed into the skin surface to levels below the maximum permissible exposure (MPE) while maintaining relatively high laser energy injection, thereby enhancing the strength of photoacoustic signals elicited from illuminated tissues. In one aspect, a fiber tip diffuser that includes 10 μm silica microspheres enabled relatively extensive scattering accompanied by minimal output energy loss (<5% loss). In another aspect, light delivery to tissues using the disclosed optic fiber tip diffuser may enhance light delivery to tissue by as much as six-fold over a fiber tip end face while keeping light fluence below MPE. In various aspects, systems, methods, and devices that include the disclosed fiber tip end diffuser are suitable for use in a variety of applications including, but not limited to, endo-cavity photoacoustic imaging.

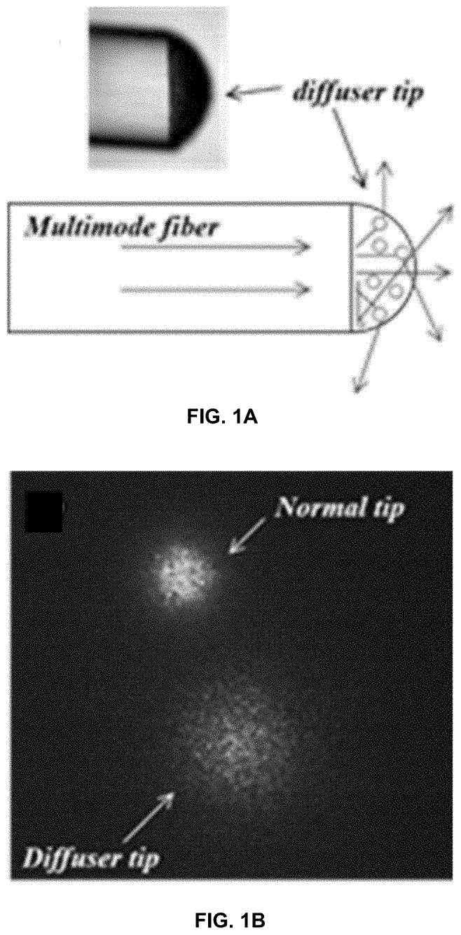

The fiber tip diffuser, as depicted in FIG. 1 (a), is made of silica microspheres (10 μm diameter, refractive index=1.458) mixed within ultraviolet (UV) adhesive (Norland Optical Adhesive 63, refractive index=1.56). To produce the fiber tip diffuser, a fiber end facet is polished, and then dipped into a microsphere-adhesive mixture. A hemispherical tip is thus formed on the fiber end facet under surface tension of the adhesive. After curing for several minutes under UV light exposure, the fiber tip diffuser is formed. Light photons entering the hemispherical tip are subjected to a Mie scattering process generated by the refractive index difference between the microspheres and surrounding UV adhesive, thus scattering the light photons and reducing output fluence.

In various aspects, the diffuser concentration is calculated as the ratio between the mass of UV adhesive and microspheres. For example, a 20:10 diffuser is produced using a mixture of 0.20 grams UV adhesive and 0.10 grams silica microspheres. The higher concentration of silica microspheres enhances the number of scattering events the light photons are subjected to, resulting in a higher degree of light diffusion at the output end. However, by mixing more and more silica microspheres inside the UV adhesive, the solution becomes saturated with silica microspheres. In one aspect, a 20:10 fiber diffuser tip is the highest microspheres concentration for which a hemispherical diffuser tip may be produced using the methods described herein. Without being limited to any particular theory, diffuser concentrations higher than 20:10 are likely to result in solidification of microspheres in the UV adhesive.

### CNN Architecture

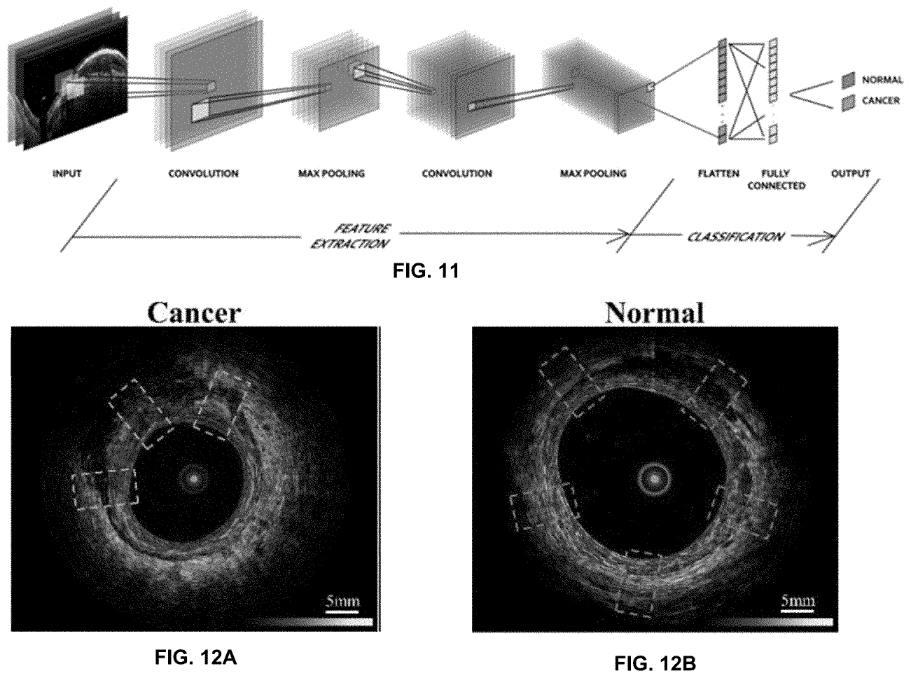

In various aspects, the overall architecture of the CNN is configured to distinguish normal from malignant colorectal tissue based on US and PA images obtained using the US-PAM probe described herein. In some aspects, separate neural networks are produced for ultrasound (US-CNN) and co-registered PA images (PAE-CNN). As illustrated in FIG. 11, both the PAE-CNN and US-CNN contain two sequential feature extraction layers and two fully connected layers. Each feature extraction layer includes one convolutional layer, followed by one pooling layer. Each convolutional layer uses a 3×3 kernel, and each pooling layer has a 2×2 kernel with max-pooling. The first fully connected layer following the feature extraction layers is a hidden layer with 512 nodes. The second fully connected layer is the output layer, which has only two outputs, corresponding to either a normal colorectal tissue, referred to as a layered tissue structure for US and a layer-like vascular distribution for PAE, or to abnormal or malignant colorectal tissue. The output layer has a ‘softmax’ activation function which predicts the probability of an input image being in a certain class (e.g., a layered tissue structure for US and layer-like vascular distribution for PAE). In some aspects, the CNN assigns each input image a probability of normal (equal to 1 minus the probability of malignancy). All other layers have ‘ReLU’ activation, which immediately sets all negative values to zero. A ReLU activation function with a gradient of 1 for positive inputs and 0 for negative inputs prevents the occurrence of exploding or vanishing gradient problems.

### Computing Systems and Devices

In various aspects, the AR-PAE/US system further includes a computing device (FIG. 31) operatively coupled to at least one element of the AR-PAE/US system including, but not limited to, the function generator, the pulser/receiver, the Nd-YAG generator, and one or more elements of the imaging probe including, but not limited to, the ultrasound transducer and stepper motors or other actuators. In some aspects, the computing device is configured to control one or more aspects of the operation of the AR-PAE/US system including, but not limited to, coordinating the operation of the laser and US transducer to implement co-registered PA and US imaging, US and PA trigger signals, controlling the operational parameters of the pulser receiver, controlling the operational parameters of the laser, controlling the operation of the function generator. In some aspects, the computing device may include software that performs the function of one or more elements of the AR-PAE/US system such that one or more elements may be omitted from the AR-PAE/US system. In various aspects, the computing device may replace the function of at least one system element including, but not limited to, the function generator and the pulser receiver.

In various other aspects, the computing device of the AR-PAE/US system is further configured to perform data acquisition (DAQ), process the PA and US signal data from the imaging probe to produce co-registered PA and US images, and to classify the US and/or PA images as normal or cancerous tissue using the deep learning CNN model as described herein.

In some aspects, the computing device is configured to produce a display to a practioner. The display may include any relevant information useful to the practitioner to facilitate surgical planning, to assess a patient's response to treatment, to select a treatment for the patient, and any other relevant information without limitation. Non-limiting examples of suitable information included in the display include co-registered PA and US images, PA flowmetric data maps, probabilities of imaged tissue being normal or cancerous, and any other information without limitation.

By way of non-limiting example, PA and US imaging data are acquired from Labview and the display is processed in Python. One co-registered B-scan of PAM and US takes one second for data acquisition, but a few seconds are needed to jointly display both PAM and US. Real-time imaging during the exam is critical to allow surgeons to orient the probe and assess the lesion area. In one aspect, data acquisition and processing are implemented using C++ code, to implement real-time 3-D volume rendering software using co-registered PAM and US images. In some aspects, a trained PAM-CNN or US-CNN model is implemented into the real-time imaging display to produce and display probabilities of the imaged tissue being normal.

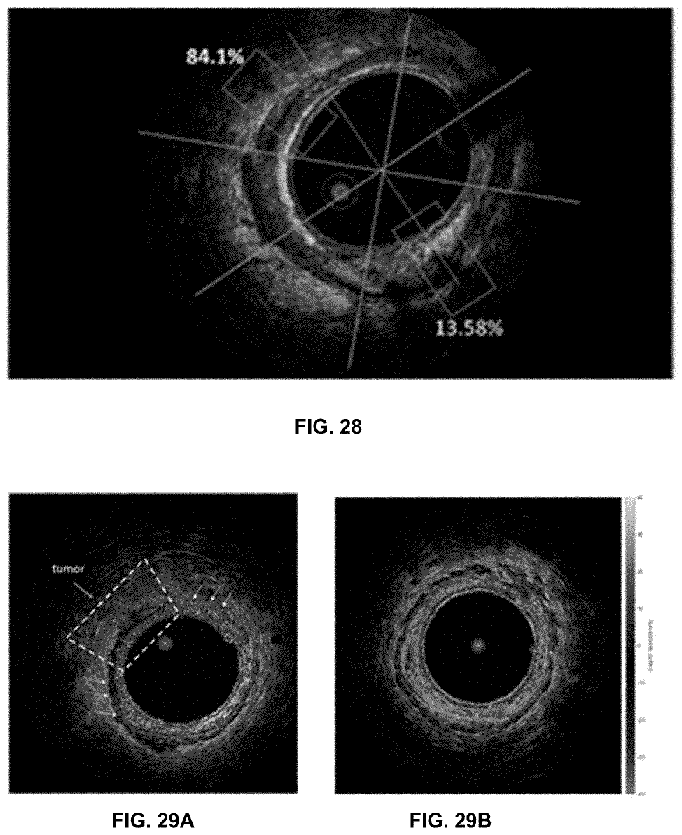

In some aspects, the CNN tissue classification is performed offline after data collection. In some other aspects, an extensively trained and validated PAM-CNN and/or US-CNN model is incorporated into the 3-D volume rendering software to provide surgeons with immediate feedback on diagnostic results. By way of non-limiting example, the automated scanning and diagnosis initially starts from 8 sectors identified using an ellipsoid fitting and segmentation code (see FIG. 28), and PAM-CNN probabilities of normal are displayed on each of these 8 sectors. These sectors can be automatically rotated based on suspicious tumor bed locations and probability outputs will follow. Neighboring sectors can be combined to provide an average probability output. In some aspects, the data processing further includes manual selection of any suspicious ROIs and output probabilities of classification, as shown in FIG. 28. In various aspects, the data processing software may be further refined based on feedbacks from surgeons.

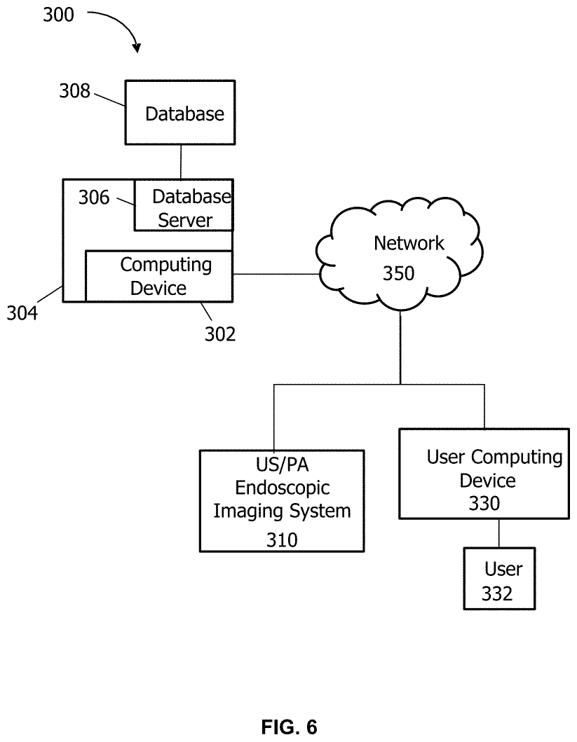

FIG. 6 depicts a simplified block diagram of a computing device 300 for implementing the methods described herein. As illustrated in FIG. 6, the computing device 300 may be configured to implement at least a portion of the tasks associated with disclosed method using the system 310 including, but not limited to: operating the ultrasound (US)/photoacoustic (PA) endoscopic imaging system 310 to obtain co-registered US and PA images of a rectal tissue of a subject and analyzing the US and PA images using the methods described herein to assess the probability that a region of interest within the US and PA images contain healthy rectal tissues. The computer system 300 may include a computing device 302. In one aspect, the computing device 302 is part of a server system 304, which also includes a database server 306. The computing device 302 is in communication with a database 308 through the database server 306. The computing device 302 is communicably coupled to the US/PA endoscopic imaging system 310 and a user computing device 330 through a network 350. The network 350 may be any network that allows local area or wide area communication between the devices. For example, the network 350 may allow communicative coupling to the Internet through at least one of many interfaces including, but not limited to, at least one of a network, such as the Internet, a local area network (LAN), a wide area network (WAN), an integrated services digital network (ISDN), a dial-up-connection, a digital subscriber line (DSL), a cellular phone connection, and a cable modem. The user computing device 330 may be any device capable of accessing the Internet including, but not limited to, a desktop computer, a laptop computer, a personal digital assistant (PDA), a cellular phone, a smartphone, a tablet, a phablet, wearable electronics, smart watch, or other web-based connectable equipment or mobile devices.

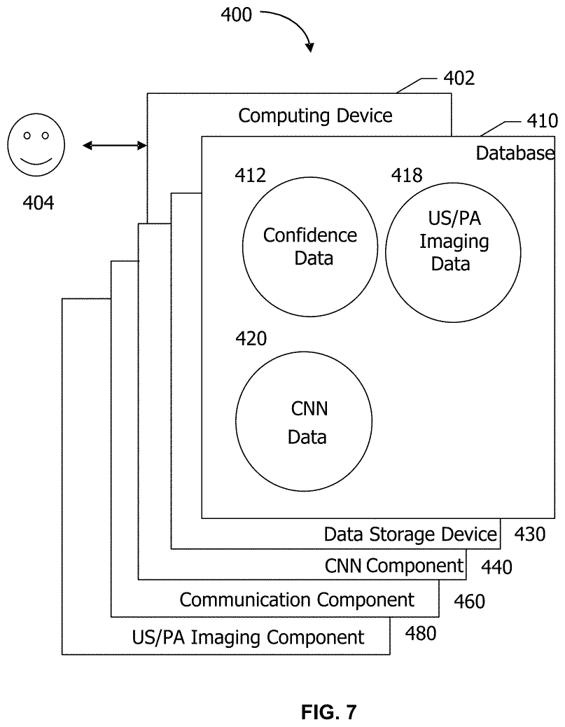

In other aspects, the computing device 302 is configured to perform a plurality of tasks associated with assessing confidence in localizations in SMLM images using a Wasserstein-induced flux (WIF) method as described herein. FIG. 7 depicts a component configuration 400 of computing device 402, which includes database 410 along with other related computing components. In some aspects, computing device 402 is similar to computing device 302 (shown in FIG. 6). A user 404 may access components of computing device 402. In some aspects, database 410 is similar to database 308 (shown in FIG. 6).

In one aspect, database 410 includes ultrasound/photoacoustic imaging data 418, convolutional computational network (CNN) algorithm data 420, and confidence data 412 defining the confidence of localizations within the SMLM imaging data. Non-limiting examples of suitable algorithm data 420 include any values of parameters defining at least one CNN model, such as any of the CNN parameters described herein. Non-limiting examples of CNN models include a CNN-US model that analyzes only US images, a CNN-PA model that analyzes only PA images, and a CNN-US/PA model that analyzes combined imaging data from the co-registered PA and US images obtained using the endoscopic US/PA imaging system as described herein.

Computing device 402 also includes a number of components which perform specific tasks. In the example aspect, computing device 402 includes data storage device 430, CNN component 440, communication component 460, and US/PA imaging component 480. Data storage device 430 is configured to store data received or generated by computing device 402, such as any of the data stored in database 410 or any outputs of processes implemented by any component of computing device 402. CNN component 440 is configured to produce a probability of normal rectal tissue composition within a region of interest of a PA and/or US image using the method described herein in various aspects. US/PA imaging component 480 is configured to operate the endoscopic US/PA imaging system as described herein to obtain co-registered US and PA images of the rectal tissue of a subject.

Communication component 460 is configured to enable communications between computing device 402 and other devices (e.g. user computing device 330 and US/PA endoscopic imaging system 310, shown in FIG. 6) over a network, such as network 350 (shown in FIG. 6), or a plurality of network connections using predefined network protocols such as TCP/IP (Transmission Control Protocol/Internet Protocol).

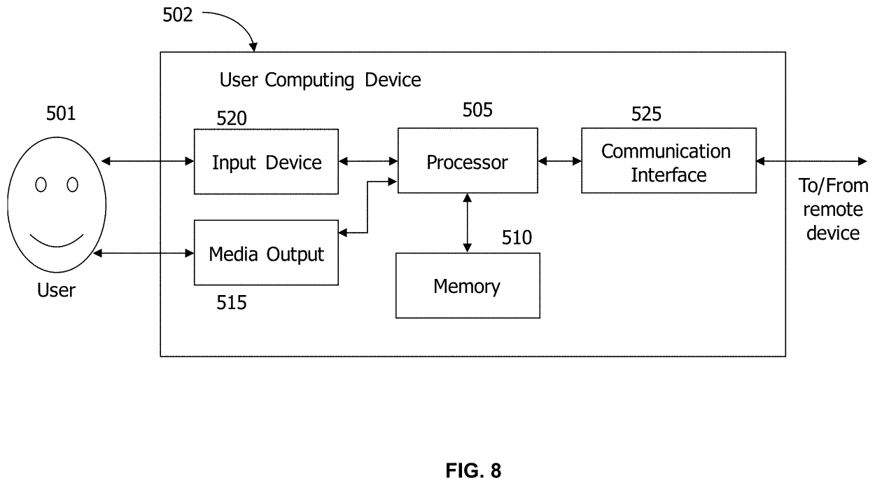

FIG. 8 depicts a configuration of a remote or user computing device 502, such as user computing device 330 (shown in FIG. 6). Computing device 502 may include a processor 505 for executing instructions. In some aspects, executable instructions may be stored in a memory area 510. Processor 505 may include one or more processing units (e.g., in a multi-core configuration). Memory area 510 may be any device allowing information such as executable instructions and/or other data to be stored and retrieved. Memory area 510 may include one or more computer-readable media.

Computing device 502 may also include at least one media output component 515 for presenting information to a user 501. Media output component 515 may be any component capable of conveying information to user 501. In some aspects, media output component 515 may include an output adapter, such as a video adapter and/or an audio adapter. An output adapter may be operatively coupled to processor 505 and operatively coupleable to an output device such as a display device (e.g., a liquid crystal display (LCD), organic light emitting diode (OLED) display, cathode ray tube (CRT), or “electronic ink” display) or an audio output device (e.g., a speaker or headphones). In some aspects, media output component 515 may be configured to present an interactive user interface (e.g., a web browser or client application) to user 501.

In some aspects, computing device 502 may include an input device 520 for receiving input from user 501. Input device 520 may include, for example, a keyboard, a pointing device, a mouse, a stylus, a touch sensitive panel (e.g., a touch pad or a touch screen), a camera, a gyroscope, an accelerometer, a position detector, and/or an audio input device. A single component such as a touch screen may function as both an output device of media output component 515 and input device 520.

Computing device 502 may also include a communication interface 525, which may be communicatively coupleable to a remote device. Communication interface 525 may include, for example, a wired or wireless network adapter or a wireless data transceiver for use with a mobile phone network (e.g., Global System for Mobile communications (GSM), 3G, 4G or Bluetooth) or other mobile data network (e.g., Worldwide Interoperability for Microwave Access (WIMAX)).

Stored in memory area 510 are, for example, computer-readable instructions for providing a user interface to user 501 via media output component 515 and, optionally, receiving and processing input from input device 520. A user interface may include, among other possibilities, a web browser and client application. Web browsers enable users 501 to display and interact with media and other information typically embedded on a web page or a website from a web server. A client application allows users 501 to interact with a server application associated with, for example, a vendor or business.

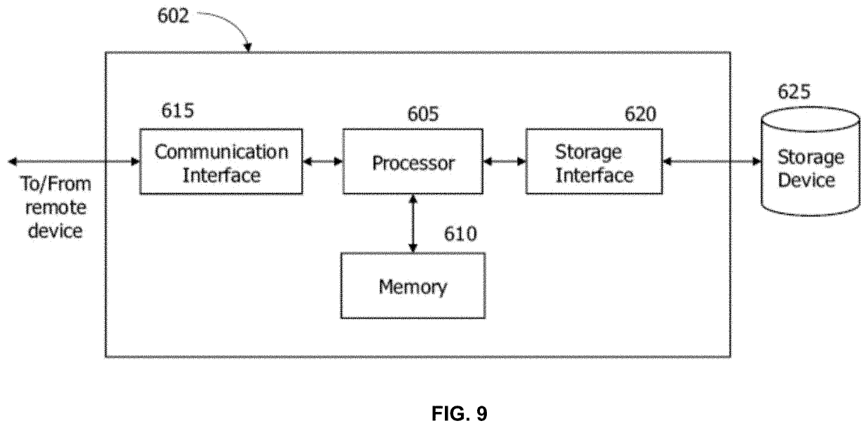

FIG. 9 illustrates an example configuration of a server system 602. Server system 602 may include, but is not limited to, database server 306 and computing device 302 (both shown in FIG. 6). In some aspects, server system 602 is similar to server system 304 (shown in FIG. 6). Server system 602 may include a processor 605 for executing instructions. Instructions may be stored in a memory area 625, for example. Processor 605 may include one or more processing units (e.g., in a multi-core configuration).

Processor 605 may be operatively coupled to a communication interface 615 such that server system 602 may be capable of communicating with a remote device such as user computing device 330 (shown in FIG. 6) or another server system 602. For example, communication interface 615 may receive requests from user computing device 330 via a network 350 (shown in FIG. 6).

Processor 605 may also be operatively coupled to a storage device 625. Storage device 625 may be any computer-operated hardware suitable for storing and/or retrieving data. In some aspects, storage device 625 may be integrated in server system 602. For example, server system 602 may include one or more hard disk drives as storage device 625. In other aspects, storage device 625 may be external to server system 602 and may be accessed by a plurality of server systems 602. For example, storage device 625 may include multiple storage units such as hard disks or solid state disks in a redundant array of inexpensive disks (RAID) configuration. Storage device 625 may include a storage area network (SAN) and/or a network attached storage (NAS) system.

In some aspects, processor 605 may be operatively coupled to storage device 625 via a storage interface 620. Storage interface 620 may be any component capable of providing processor 605 with access to storage device 625. Storage interface 620 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 605 with access to storage device 625.

Memory areas 510 (shown in FIG. 8) and 610 may include, but are not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are examples only, and are thus not limiting as to the types of memory usable for storage of a computer program.

The computer systems and computer-implemented methods discussed herein may include additional, less, or alternate actions and/or functionalities, including those discussed elsewhere herein. The computer systems may include or be implemented via computer-executable instructions stored on non-transitory computer-readable media. The methods may be implemented via one or more local or remote processors, transceivers, servers, and/or sensors (such as processors, transceivers, servers, and/or sensors mounted on vehicle or mobile devices, or associated with smart infrastructure or remote servers), and/or via computer executable instructions stored on non-transitory computer-readable media or medium.

In some aspects, a computing device is configured to implement machine learning, such that the computing device “learns” to analyze, organize, and/or process data without being explicitly programmed. Machine learning may be implemented through machine learning (ML) methods and algorithms. In one aspect, a machine learning (ML) module is configured to implement ML methods and algorithms. In some aspects, ML methods and algorithms are applied to data inputs and generate machine learning (ML) outputs. Data inputs may include but are not limited to: images or frames of a video, object characteristics, and object categorizations. Data inputs may further include: sensor data, image data, video data, telematics data, authentication data, authorization data, security data, mobile device data, geolocation information, transaction data, personal identification data, financial data, usage data, weather pattern data, “big data” sets, and/or user preference data. ML outputs may include but are not limited to: a tracked shape output, categorization of an object, categorization of a type of motion, a diagnosis based on motion of an object, motion analysis of an object, and trained model parameters ML outputs may further include: speech recognition, image or video recognition, medical diagnoses, statistical or financial models, autonomous vehicle decision-making models, robotics behavior modeling, fraud detection analysis, user recommendations and personalization, game AI, skill acquisition, targeted marketing, big data visualization, weather forecasting, and/or information extracted about a computer device, a user, a home, a vehicle, or a party of a transaction. In some aspects, data inputs may include certain ML outputs.

In some aspects, at least one of a plurality of ML methods and algorithms may be applied, which may include but are not limited to: linear or logistic regression, instance-based algorithms, regularization algorithms, decision trees, Bayesian networks, cluster analysis, association rule learning, artificial neural networks, deep learning, dimensionality reduction, and support vector machines. In various aspects, the implemented ML methods and algorithms are directed toward at least one of a plurality of categorizations of machine learning, such as supervised learning, unsupervised learning, and reinforcement learning.

In one aspect, ML methods and algorithms are directed toward supervised learning, which involves identifying patterns in existing data to make predictions about subsequently received data. Specifically, ML methods and algorithms directed toward supervised learning are “trained” through training data, which includes example inputs and associated example outputs. Based on the training data, the ML methods and algorithms may generate a predictive function which maps outputs to inputs and utilize the predictive function to generate ML outputs based on data inputs. The example inputs and example outputs of the training data may include any of the data inputs or ML outputs described above.

In another aspect, ML methods and algorithms are directed toward unsupervised learning, which involves finding meaningful relationships in unorganized data. Unlike supervised learning, unsupervised learning does not involve user-initiated training based on example inputs with associated outputs. Rather, in unsupervised learning, unlabeled data, which may be any combination of data inputs and/or ML outputs as described above, is organized according to an algorithm-determined relationship.

In yet another aspect, ML methods and algorithms are directed toward reinforcement learning, which involves optimizing outputs based on feedback from a reward signal. Specifically ML methods and algorithms directed toward reinforcement learning may receive a user-defined reward signal definition, receive a data input, utilize a decision-making model to generate a ML output based on the data input, receive a reward signal based on the reward signal definition and the ML output, and alter the decision-making model so as to receive a stronger reward signal for subsequently generated ML outputs. The reward signal definition may be based on any of the data inputs or ML outputs described above. In one aspect, a ML module implements reinforcement learning in a user recommendation application. The ML module may utilize a decision-making model to generate a ranked list of options based on user information received from the user and may further receive selection data based on a user selection of one of the ranked options. A reward signal may be generated based on comparing the selection data to the ranking of the selected option. The ML module may update the decision-making model such that subsequently generated rankings more accurately predict a user selection.

As will be appreciated based upon the foregoing specification, the above-described aspects of the disclosure may be implemented using computer programming or engineering techniques including computer software, firmware, hardware or any combination or subset thereof. Any such resulting program, having computer-readable code means, may be embodied or provided within one or more computer-readable media, thereby making a computer program product, i.e., an article of manufacture, according to the discussed aspects of the disclosure. The computer-readable media may be, for example, but is not limited to, a fixed (hard) drive, diskette, optical disk, magnetic tape, semiconductor memory such as read-only memory (ROM), and/or any transmitting/receiving medium, such as the Internet or other communication network or link. The article of manufacture containing the computer code may be made and/or used by executing the code directly from one medium, by copying the code from one medium to another medium, or by transmitting the code over a network.

These computer programs (also known as programs, software, software applications, “apps”, or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The “machine-readable medium” and “computer-readable medium,” however, do not include transitory signals. The term “machine-readable signal” refers to any signal used to provide machine instructions and/or data to a programmable processor.

As used herein, a processor may include any programmable system including systems using micro-controllers, reduced instruction set circuits (RISC), application specific integrated circuits (ASICs), logic circuits, and any other circuit or processor capable of executing the functions described herein. The above examples are examples only, and are thus not intended to limit in any way the definition and/or meaning of the term “processor.”

As used herein, the terms “software” and “firmware” are interchangeable, and include any computer program stored in memory for execution by a processor, including RAM memory, ROM memory, EPROM memory, EEPROM memory, and non-volatile RAM (NVRAM) memory. The above memory types are example only, and are thus not limiting as to the types of memory usable for storage of a computer program.

In one aspect, a computer program is provided, and the program is embodied on a computer readable medium. In one aspect, the system is executed on a single computer system, without requiring a connection to a sever computer. In a further aspect, the system is being run in a Windows® environment (Windows is a registered trademark of Microsoft Corporation, Redmond, Wash.). In yet another aspect, the system is run on a mainframe environment and a UNIX® server environment (UNIX is a registered trademark of X/Open Company Limited located in Reading, Berkshire, United Kingdom). The application is flexible and designed to run in various different environments without compromising any major functionality.

In some aspects, the system includes multiple components distributed among a plurality of computing devices. One or more components may be in the form of computer-executable instructions embodied in a computer-readable medium. The systems and processes are not limited to the specific aspects described herein. In addition, components of each system and each process can be practiced independent and separate from other components and processes described herein. Each component and process can also be used in combination with other assembly packages and processes. The present aspects may enhance the functionality and functioning of computers and/or computer systems.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Any publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following examples illustrate various aspects of the disclosure.

### Example 1: Co-Registered PAM-US Imaging Probe and System Development

To develop and refine a combined photoacoustic microscopy and ultrasound (PAM-US) probe capable of obtaining co-registered PAM and ultrasound images suitable for colorectal cancer assessment, the following experiments were conducted.

The PAM-US imaging probe used in these experiments for ex vivo imaging studies is shown illustrated in FIGS. 21A, 21B, and 21C. As illustrated in FIG. 21A, a multi-mode fiber (1.5 mm diameter, numerical aperture of 0.39) delivered 750 nm laser pulses to the imaging head. At the distal end of the imaging head, a focused ring transducer (focal-depth-12.7 mm, 20 MHz, Capistrano Labs, CA) was installed on the side; a 45° rod mirror reflects the laser beam 90° to the tissue surface through a center hole in the ring transducer. The PAM-US probe described above was incorporated into the endoscope of a PAM-US system, as illustrated in FIG. 31. Light output from an Nd:YAG (Lotis TII) pumped Ti:Sapphire (15 Hz, 10 ns) laser was focused by a convex lens (f=40 mm) to a multi-mode fiber. The distal end of a multimode fiber was fixed on the endoscopic PAM probe (see FIG. 21A). A pulser/receiver (Panametrics 5900PR) was used to amplify photoacoustic (PA) signals in PA mode and obtain US images in Pulse/Echo mode. Sequentially co-registered PA and US scanning was controlled by a motor. This system took 7 seconds to finish a US B-scan and 56 seconds for a photoacoustic B-scan of 20 mm size.

To improve the resolution of the images obtained by the PAM/US system and to provide for in vivo patient imaging, the US-PAM probe was modified to produce the US-PAM probe as illustrated in FIGS. 10A, 10B, and 10C. The new endorectal probe included three parts—a handle, water channel, and imaging head (FIG. 10A). A stepper motor integrated with the handle rotated the imaging head through a transmission shaft within the water channel (FIGS. 10B and 10C). The transmission shaft and stepper motor were hollow, allowing the passage of the long optical fiber and ultrasonic transducer wire from the back of the probe to the imaging head. When in use, the probe head was covered by a latex balloon (FIG. 10C) that was inflated via a water channel accessed near the probe handle (FIG. 10B). When inflated with deionized water, the fluid enabled transmission of ultrasound and PA waves from the imaging head to the surrounding rectal wall. The design of PAM probe was based on an FDA-approved conventional endorectal ultrasound probe (BK Medical Inc. MA). With this design, the PAM/US probe fit perfectly into standard rigid proctoscopes, which were widely available, and were easily operated by surgeons familiar with performing standard endorectal US examinations.

The in vivo imaging probe head contained several components that provided for side-viewing image capture over 360 degrees (FIG. 22). A SF-11 prism reflected and coupled the laser beam at 90° to the tissue surface through a center hole in the ring transducer. The imaging probe head was incorporated into a PAM/US imaging system, shown illustrated in FIG. 31. A function generator triggered a pulser/receiver to acquire US pulse-echo signals. Following each trigger of a US A-scan, a delayed laser trigger initiated the acquisition of a PA A-scan. In this way, the handheld PAM system was able to simultaneously register US and PA data. Data acquisition speed was improved by the use of an Nd:YAG laser operated at 1064 nm with a 1 KHz repetition rate, a pulse energy of 9 mJ and a duration of 7 ns. Using the 1 kHz laser pulse repetition rate, an imaging frame rate of one B-scan image per second was achieved. A 3.6 mJ laser pulse from the probe tip illuminated a tissue area of approximately 0.15 cm², resulting in a surface optical fluence of 26 mJ/cm², which was well within the safety threshold at 1064 nm.

### Example 2: Ex Vivo Imaging Using Co-Registered PAM-US Imaging Probe

To evaluate the ex vivo imaging of the combined photoacoustic microscopy and ultrasound (PAM-US) probe described herein, the following experiments were conducted.

Colorectal specimens were collected from subjects with normal bowel tissue, colon cancer patients previously treated with chemotherapy with residual disease, and rectal cancer patients treated with chemotherapy and radiation with pathological complete response (pCR). Specimens were obtained from patients undergoing resection of biopsy-proven rectal cancer.

The ex vivo imaging probe described in Example 1 was used to obtain co-registered PAM-US images as described above. Each specimen was imaged fresh prior to formalin fixation. All ex vivo specimen imaging was completed within one hour of surgery and histologically analyzed per pathologic standards. For each in vivo study patient, normal and residual tumor locations were imaged prior to resection and then assessed histologically by the collaborating pathologist.

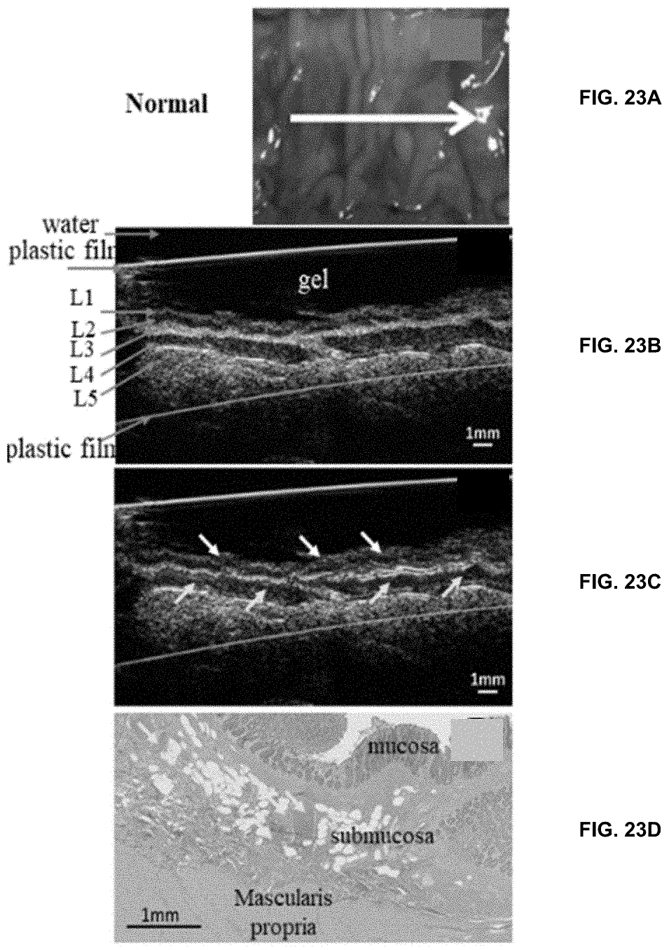

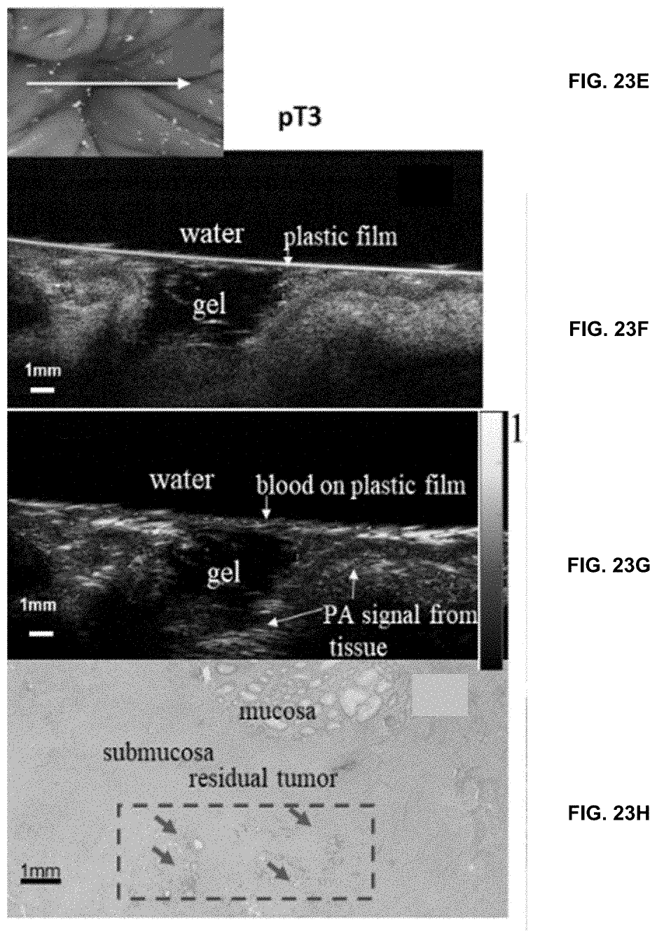

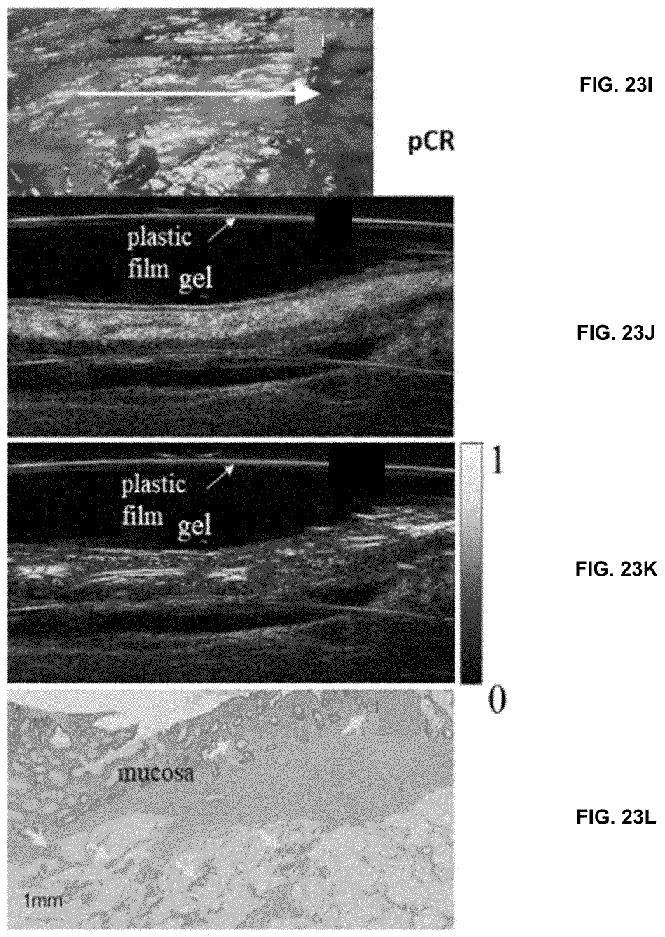

The images of FIGS. 23A, 23B, 23C, and 23E were of a specimen obtained from a segment of normal bowel. A photograph of the imaged area (FIG. 23A) was collected at the time of imaging; the white arrows indicate the plane through which the corresponding cross-sectional imaging was collected. In the solely ultrasound image, the normal layered structure of the colon wall was clearly delineated (FIG. 23B). On the surface, the first two layers (L1 and L2) represented the mucosal surface of the colon and muscularis mucosa. Under the muscularis, the basement membrane (L3) appeared echogenic (white), corresponding to the submucosa. The hypoechoic layer L4 was muscularis propria. Finally, the deepest layer was the muscularis propria (L5) and appeared as the thickest layer. FIG. 23C was the same US image now co-registered with photoacoustic signals, demonstrating areas of vascular signal within the bowel segment. Heavy concentrations of blood vessels were noted within the submucosa (indicated by high intensity color). H&E images from the same specimen correlated with the co-registered US and PAM images—the submucosa was rich in vascular structures while the underlying muscle lacks organized vessels (FIG. 23D).

The images of FIGS. 23E, 23F, 23G, and 23H were obtained from a colon cancer (T3N1 adenocarcinoma) specimen previously treated with six cycles of chemotherapy. FIG. 23E is a photograph of the ulcerated tumor bed surrounded by apparently normal mucosa. Ultrasound imaging across the lesion center demonstrated a peripheral multilayer structure that became disorganized within the actual tumor bed (the central portion of FIG. 23F beneath the gel-filled ulcer cavity). Similarly, the co-registered photoacoustic image (FIG. 23G) also appeared relatively normal in the periphery; however, the regular photoacoustic signal distribution pattern was transformed into hyperintense areas of high signal immediately around and below the ulceration. Corresponding to the photoacoustic findings, histologic evaluation found a significant amount of residual cancer around the ulcerated cavity (FIG. 23H). In essence, the photoacoustic signal pattern appeared to correlate with the malignant tissue found in proximity to the ulcer cavity. These images suggested that chemotherapy, in the absence of complete tumor death, did not reverse the alterations in the PAM signal noted in malignant tissue.

The images of FIGS. 231, 23J, 23K, and 23L were obtained from a rectal cancer specimen that had been treated with radiation and chemotherapy before surgical resection. Gross evaluation demonstrated a pale, firm scar (FIG. 23I). Unlike the previously described cancer specimens, both ultrasound and photoacoustic images yielded a regularly layered structure with normal vascular distribution in the submucosa (FIGS. 23J and 23K). These images appeared much more similar to the patterns noted in the normal tissue specimens (FIGS. 23B and 23C). Subsequent histologic evaluation of the specimen revealed no viable cancer cells; instead, the preoperative radiation and chemotherapy had completely destroyed the malignancy. Only fibrous scar tissue and mucin pools delineated the area of prior cancer (FIG. 23L). Blood vessels, labeled by yellow arrows, were noted throughout the submucosa. Complete tumor death after treatment resulted in tissue that produced a normal-appearing PAM signal. Significantly, scar tissue formed by the dead tumor did not appear to interfere with the return of a normal vascular signal to these tissue areas. This finding suggested a potential method for differentiating incomplete versus complete tumor responses.

### Example 3: In Vivo Imaging Using Co-Registered PAM-US Imaging Probe

To evaluate the in vivo imaging of the combined photoacoustic microscopy and ultrasound (PAM-US) probe described herein, the following experiments were conducted.

Colorectal images were obtained using the in vivo PAM-US probe described in Example 1 incorporated into the system described above and illustrated in FIG. 31. The colorectal images were obtained from patients with rectal cancer patients within a tumor-containing region and within a more distal region of healthy tissue.

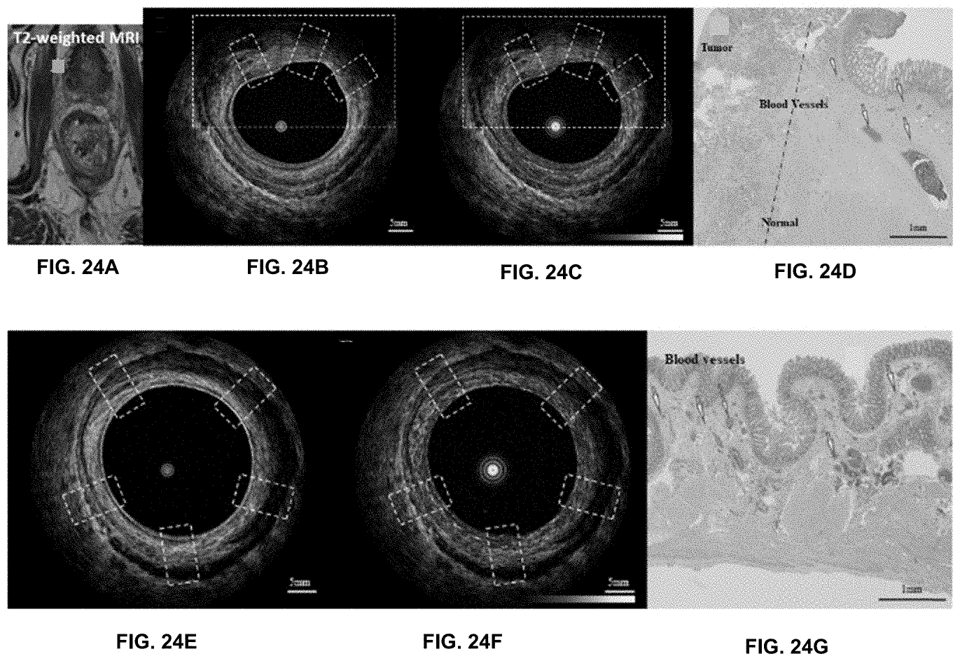

FIGS. 24A, 24B, 24C, 24D, 24E, 24F, and 24G were obtained from a patient with highly invasive rectal cancer treated with chemotherapy and radiation. Images were obtained prior to a large pelvic resection. Post-treatment MRI images showed an anterior tumor with T2 hyperintensity suggestive of residual disease and a region of low signal with regular diffusion in the posterior rectum, indicating normal tissue. PAM showed a distorted morphology in the region associated with the MRI-identified tumor (marked by the white box in FIG. 24A). PA signals were recorded at the balloon-rectal surface interface along the suspected tumor; the tumor bed was otherwise remarkable only for intermittent, poorly organized islands of signal. At resection, the mucosal tissue overlying the tumor was noted to be macerated and bleeding, likely causing the high PA signal captured at the interface of the probe and bowel wall. Histologic imaging (FIG. 24C) of the tumor bed was notable for a large residual cancer (pT3) penetrating the rectum. While the tissue surrounding the tumor was hypervascular (vessels denoted by arrows), the cancer itself was devoid of organized microvascular network as previously seen in ex vivo studies.

FIGS. 24D and 24E were obtained from the distal end of the same rectum containing normal tissue. The layered structure characteristic of normal tissue was observed in the US image (FIG. 24D) and the layer-like vascular pattern was observed in the PA image (FIG. 24E). H&E images indicated normal submucosa with rich blood vessels (FIG. 24F).

### Example 4: Development and Evaluation of Deep-Learning Convolution Neural Network (CNN) for Identifying Healthy and Cancerous Colon Tissues

To develop and evaluate CNN models for identifying healthy and cancerous tissues within the images obtained by the PAM-US imaging system described herein, the following experiments were conducted.

Prior to development of the CNN models as described herein, histograms of PA signals and the spectral slopes and intercepts of PA frequency domain data were evaluated and no features were identified that robustly separated normal colorectal tissue from residual cancer with statistical significance. However, the unique layered structures observed in US images of normal colorectal tissue and the layer-like vascular patterns in PAM images of normal colorectal tissue, as well as the recovered vascular patterns observed in corresponding images from responders to colorectal cancer treatments motivated the development of a deep-learning CNN model to can capture these unique patterns for use in identifying healthy from cancerous colorectal tissues.

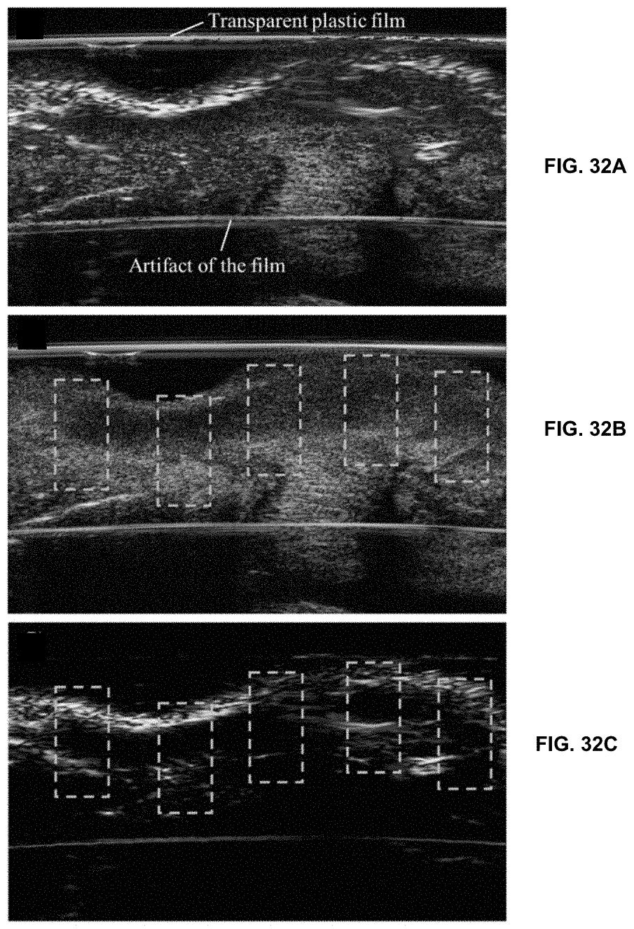

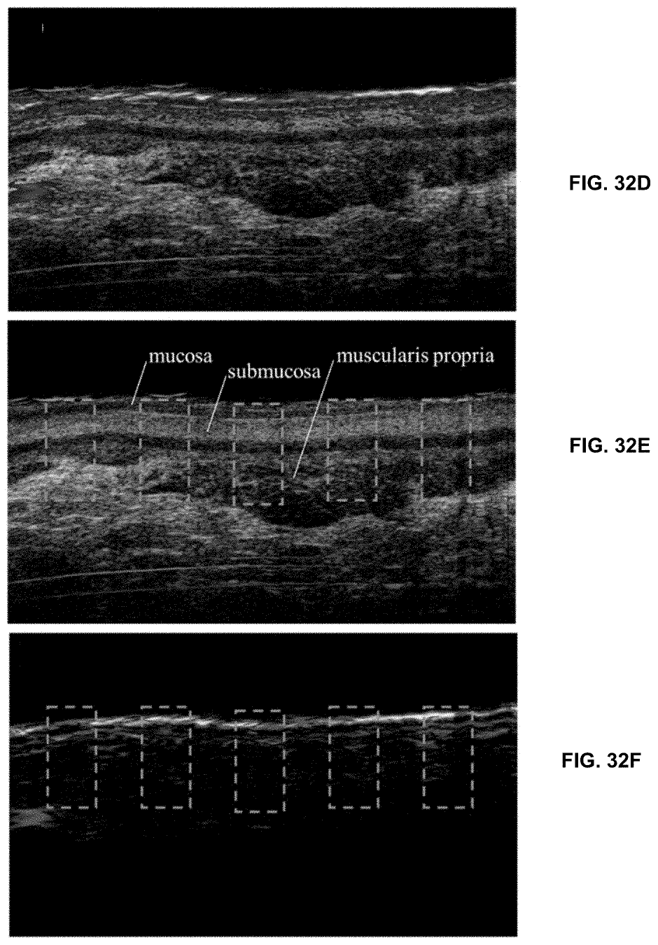

Ex-vivo normal and malignant colorectal tissue images from 22 patients similar to those obtained in Example 2 were used to train CNNs. Additionally, in vivo data obtained from two patients as described in Example 3 were also used to train the CNNs. Multiple US and AR-PAM B-scans were acquired from each specimen. In each image, 3 to 5 regions of interest (ROIs) were selected, shown as dashed-line rectangles overlaid on the US and PAM images of FIGS. 24B, 24C, 24E, and 24F. Normal tissue ROIs from US (FIGS. 24D and 32E) showed a clear layered structure, however, malignant tissue ROIs showed distorted tissue structures (FIGS. 24A and 32B). PAM normal ROIs showed layer-like PA signals due to submucosa blood vessel distribution (FIGS. 24E and 32E), but malignant ROIs showed fewer blood vessels and a distorted vascular pattern in the core of the cancer region (FIGS. 24B and 32C).

To create the training sample of images, ultrasound ROIs (1090 normal and 1492 malignant) and PA ROIs (1019 normal and 1078 malignant) were compiled from 22 patients' ex vivo images and in vivo images from 2 additional patients. Each ROI measured 450×250 pixels. Despite the large number of samples, the high dimensionality of the training data resulted in overfitting and a very long training time. Since a layered structure was not a complex feature and could be detected in a relatively lower resolution image, we reduced the dimensionality of the input data by resizing each ROI to 90×50 pixels. For testing, randomly selected ROIs were extracted from the US and PAM images of treated rectal cancer cases. Those ROIs were then resized to 90×50 pixels as well. In additional experiments, the dimensionality of the input data was reduced by resizing each ROI to 45×25 pixels, which preserved the lower spatial frequency image profile and reduced the higher spatial frequency details

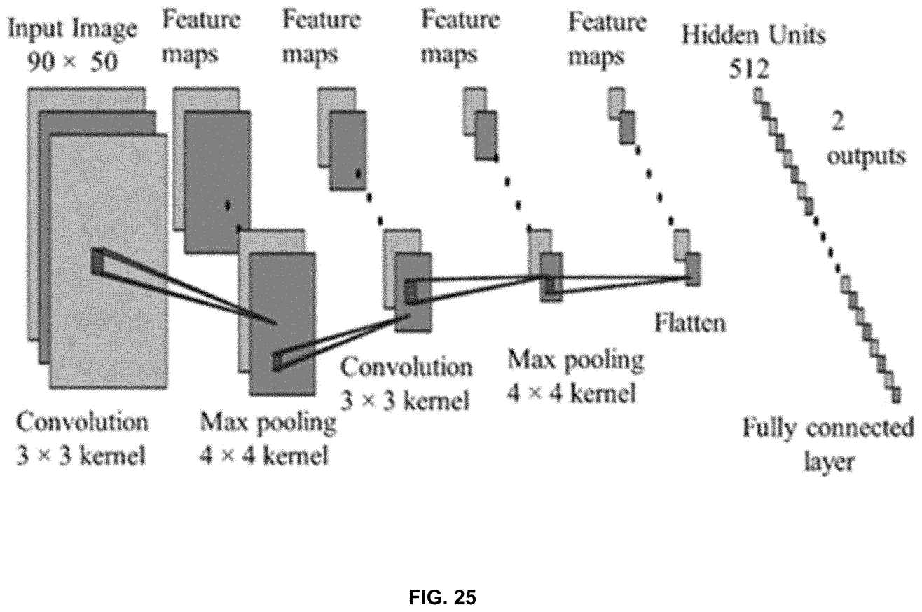

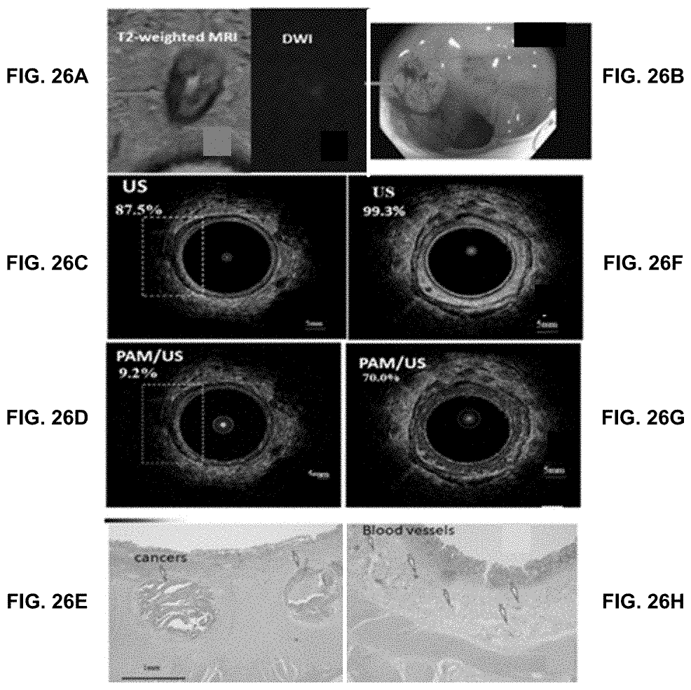

The overall architecture of the CNN model designed to distinguish a normal colorectal tissue from an abnormal or malignant tissue is depicted in FIG. 25. Briefly, the net contained two sequential feature extraction layers of one convolutional layer each, followed by one pooling layer and two fully connected layers or dense layers. Each convolutional layer used a 3×3 kernel, and each pooling layer had a 4×4 kernel with max-pooling, which extracted the maximum value from the kernel area of the feature map. Each pooling layer summarized the neighborhood kernel area of the feature map. The first fully connected layer following the feature extraction layers was a hidden layer with 512 nodes. The second fully connected layer was the output layer, which had only 2 outputs corresponding to either a layered tissue structure or corresponding normal colorectal tissue, or a non-layered tissue structure or corresponding abnormal or malignant colorectal tissue

Two CNN models with the same architecture shown in FIG. 6 were trained separately for US and PA images. Two-thirds of the total samples were used for training, and the rest were used as a validation set to tune number of epochs and learning rate. Twenty cross validations were performed (i.e., 20 models were obtained, each of which was trained on a different subset of the whole training data). The maximum number of epochs was set to 20. Validation accuracy was used as a performance metric for monitoring early stopping with a tolerance of 2 epochs (i.e. training was stopped if there was no increase in validation accuracy for two successive epochs). Stochastic gradient descent was used with a batch size of 20. Neural net weights were optimized by RMSprop optimizer, which was essentially gradient descent with momentum. The learning rate was set to 10⁻³ with a decay of 10⁻⁵. Categorical cross entropy was used as the loss function. A dropout layer was added after each pooling layer to prevent overfitting. The dropout probability was set to 0.25, which implied the output of one in every four hidden neurons were randomly set to zero. Dropped out neurons did not go forward to pass or back-propagate, so only robust features (layered or layer-like structure) were learned.

### Example 5: Testing of Deep-Learning Convolution Neural Network (CNN) for Identifying Healthy and Cancerous Colon Tissues in Ex Vivo Samples

To test the CNN models described in Example 5 for identifying healthy and cancerous tissues within the images obtained by the PAM-US imaging system described herein, the following experiments were conducted.

Ex vivo samples were obtained from patients and imaged as described in Example 2 above. Multiple ROIs were extracted from each co-registered image. Each ROI was tested using the trained PAE- and US-CNN models described in Example 4, and a probability of normal was obtained for each ROI. The average of all probabilities of normal associated with the ROIs for each case was calculated, and a threshold of 50% was used to determine if each case was normal or not. Table 1 summarizes the CNN testing results from the four cases used for testing, which included three responders with no residual tumor (R1, R2, R3) and one non-responder (NR4). The PAE-CNN classified both the responder and non-responder groups correctly, while US-CNN missed two responders.

TABLE 1
CNN Classifier tested on ex vivo treatment responder and
non-responders

| Responder | Probability of normal | | | |
|---|---|---|---|---|
| Case¹ | Photoacoustic | Ultrasound | Expected | Pathologic result |
| R1 | 98.43% | 93.68% | 100% | pCR- |
| | | | | no residual tumor |
| R2 | 96.03% | 44.41% | 100% | pCR |
| R3 | 80.04% | 14.59% | 100% | pCR |
| NR4 | 40.61% | 22.09% | 0% | Metastatic |
| | | | | adenocarcinoma |

R signifies pCR (no residual tumor); NR signifies a non-responder; Probability of normal refers to the mean probability of normal for all tested ROIs; Expected probability that responders are classified as normal is 100%, and the probability that non-responders are classified as normal is 0%.

### Example 6: Testing of Deep-Learning Convolution Neural Network (CNN) for Identifying Healthy and Cancerous Colon Tissues In Vivo

To test the CNN models described in Example 5 for identifying healthy and cancerous tissues within the images obtained by the PAM-US imaging system described herein, the following experiments were conducted.

In vivo images of four patients were obtained as described in Example 3. ROIs from four patients with colorectal cancer were evaluated using the CNN model trained using US images (US-CNN) and using the CNN model trained using PAM images (PAM-CNN). For the patients, ROIs within a tumor bed region and ROIs within a heathy colorectal region was evaluated using US-CNN and PAM-CNN to determine a probability that each ROI contained normal tissue. Table 2 summarizes the results of this evaluation.

TABLE 2
Testing results of US-CNN and PAM-CNN of 4 patients

| | Probability of normal (%) | | | Pathological |
|---|---|---|---|---|
| Patient/region | US-CNN | PAM-CNN | Expected | Results |
| #1 tumor bed | 64.1% | 20.5% | 0.0% | pT2 residual |
| #2 tumor bed | 47.0% | 23.1% | 0.0% | 1.2 recurrence |
| | | | | tumor |
| #3 tumor bed | 87.5% | 9.2% | 0.0% | pT3 residual |
| #4 tumor bed | Imaging unavailable due to rectal stricture | | | |
| #1 normal | 90.0% | 89.9% | 100% | normal |
| #2 normal | 99.3% | 70.0% | 100% | normal |
| #3 normal | 62.3% | 75.4% | 100% | normal |
| #4 normal | 71.9% | 94.2% | 100% | normal |

Probability of normal refers to the mean probability of normal for all tested ROIs; Expected probability that responders are classified as normal is 100%, and the probability that non-responders are classified as normal is 0%.

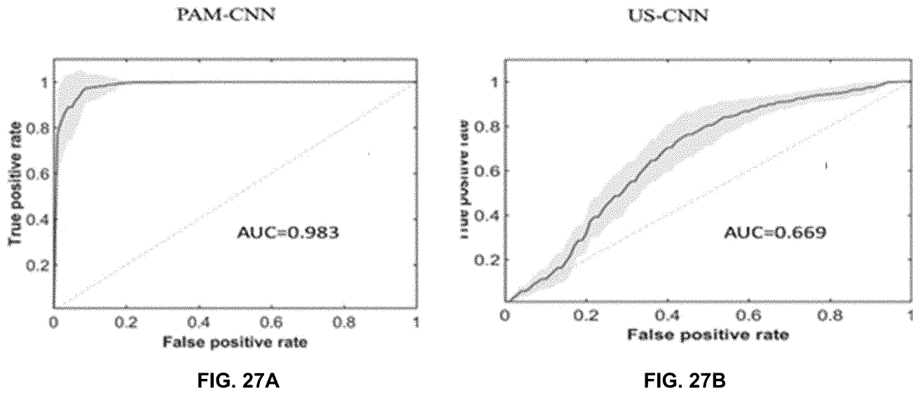

In ROIs containing histologically-confirmed malignancy, the PAM-CNN model yielded the following low average probabilities of the tissue being normal—20.5% (Patient 1), 23.1% (Patient 2), 9.2% (Patient 3). The US-CNN model produced probabilities of 64.1%, 47.0%, 87.5%, respectively. Two patients were misclassified as cancer free assuming a 50% threshold value. In normal ROIs, both PAM-CNN and US-CNN models provided correct classifications. From a total number of 132 PA ROIs and 132 US ROIs (54 malignant ROIs and 78 normal ROIs) obtained from four patients, ROCs were computed as shown in FIGS. 27A and 27B; the areas under ROC were 0.983 and 0.669 for PAM-CNN and US-CNN, respectively.

### Example 7: Testing of Deep-Learning Convolution Neural Network (CNN) for Identifying Healthy and Cancerous Colon Tissues In Vivo

To test the CNN models described in Example 5 for identifying healthy and cancerous tissues within the images obtained by the PAM-US imaging system described herein, the following experiments were conducted.

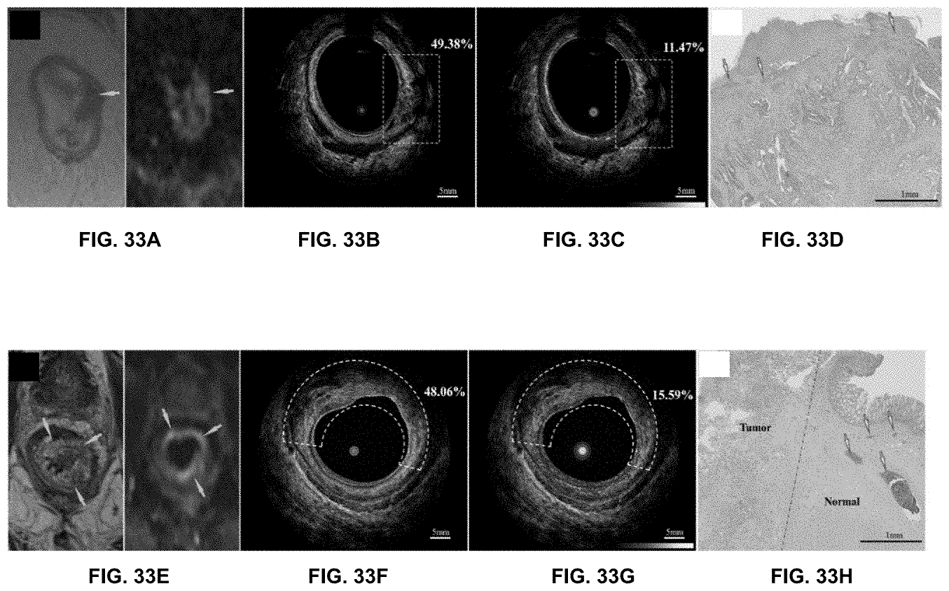

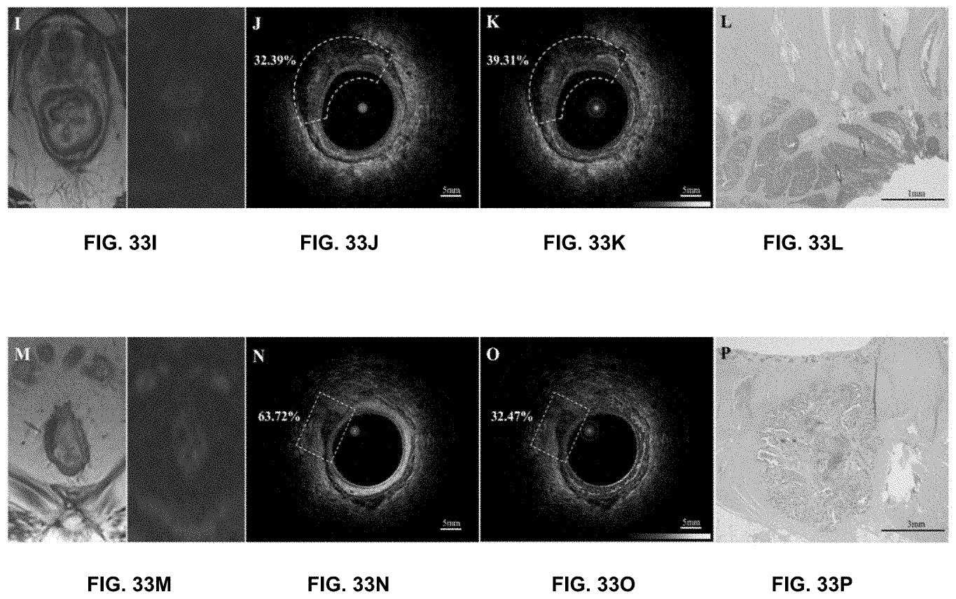

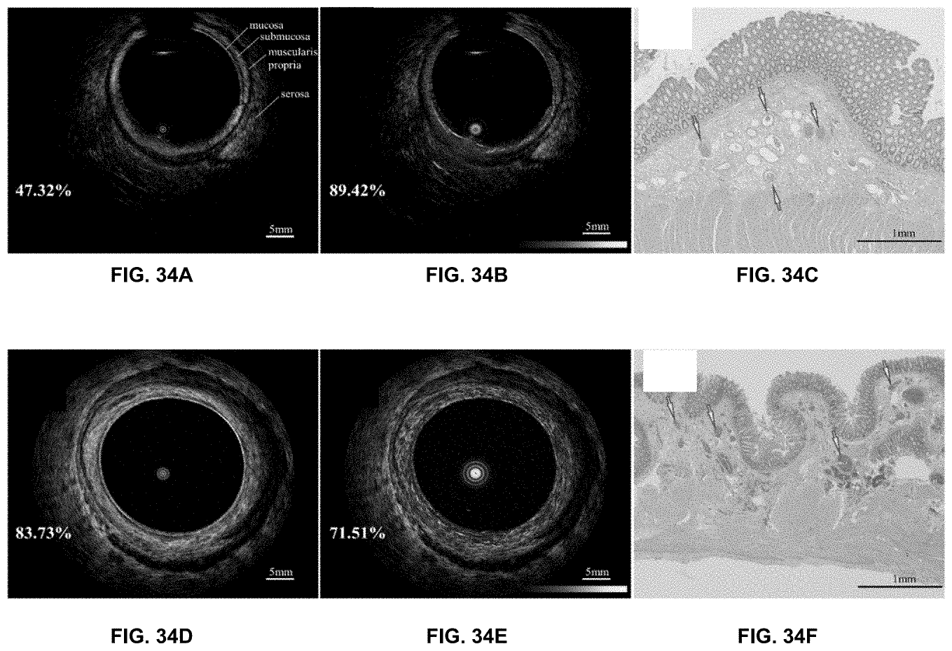

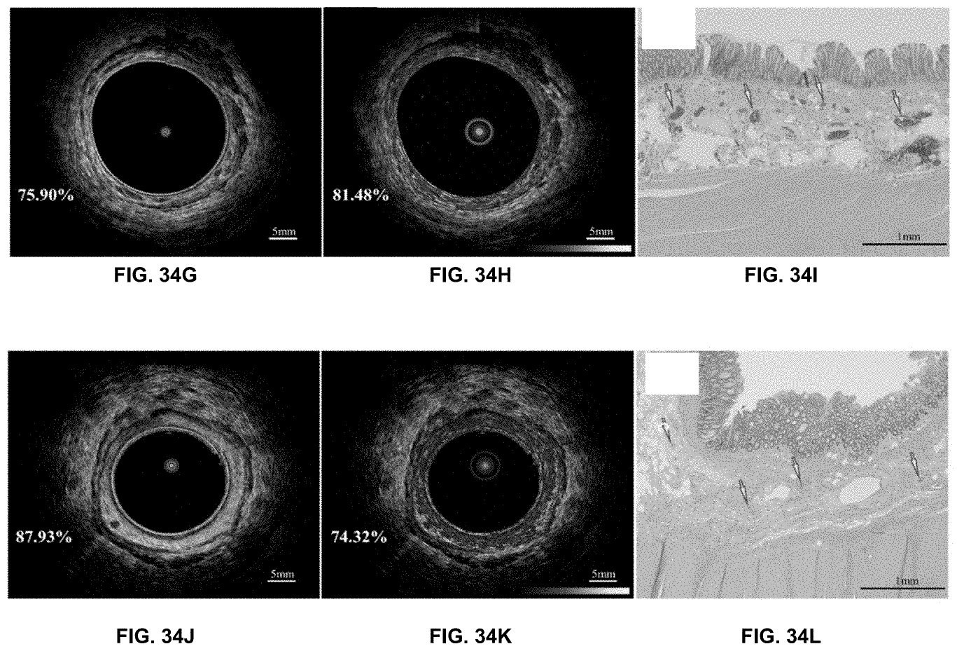

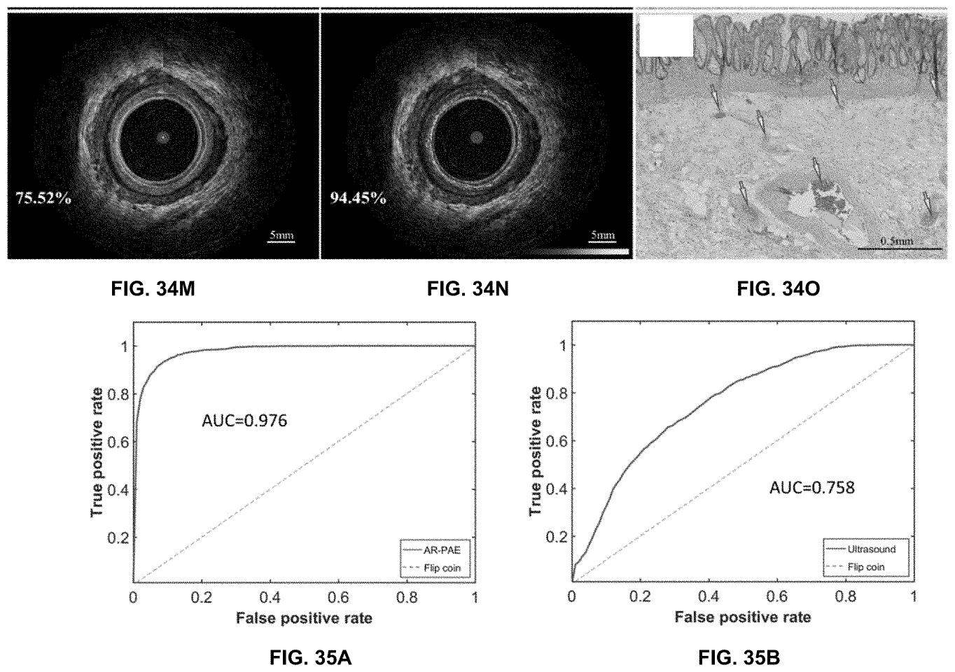

Five patients were imaged intraoperatively prior to resection. FIGS. 33A, 33B, 33C, 33E, 33F, 33G, 33I, 33J, 33K, 33M, 33N, and 33O show representative T2-weighted MRI, DWI, US, and co-registered PAE/US images taken in the residual tumor region from the patients, with images of the normal regions of the patients shown in FIGS. 34A, 34B, 34D, 34E, 34G, 34H, 34J, 34K, 34M and 34N. Due to a rectal stricture diagnosed intraoperatively in Patient #5, the tumor area could not be reached by the probe, and only a normal region close to anus was imaged (FIGS. 34M and N).

The first patient was a 61-year-old woman with rectal cancer treated with chemotherapy and radiation. Low anterior resection was performed to remove the sigmoid colon and rectum. FIGS. 33A, 33B, 33C, and 33D show representative T2-weighted MRI, DWI, US, co-registered PAE/US, and H&E images, respectively, collected from the residual tumor region of the rectum. The MRI T2-weighted image showed intermediate signal intensity of the cancer region, as marked by the arrow (brighter than fluid, darker than muscle), and the DWI showed hyperintensity corresponding to diffusion restriction in the cancer region. The US image showed distorted morphology in the tumor area, as marked by the white box and normal layered structure outside the box. A black area at 12 o'clock of the US image is due to a small air bubble remaining in the ultrasound balloon during imaging, which blocked the ultrasound signal. The PA image superimposed on the co-registered US image is presented in FIG. 33C. The disrupted submucosa vascular distribution with a minimal PA signal was observed within the tumor area, while the normal submucosa had a uniformly distributed PA signal. The tumor location revealed by the PAE/US image was consistent with the T2-weighted MRI and DWI. The histology of the tumor area (FIG. 33D) showed a few tiny vessels at the surface (indicated by arrows), while the majority of the tumor region lacked blood vessels. Pathologic evaluation revealed a 1.8 cm residual rectal tumor which invaded the muscularis propria, a post-treatment stage 2 tumor (pT2). FIGS. 34A, 34B, and 34C are images of the normal bowel wall taken at the distal end of the tumor, confirmed from the resection specimen. A typical layered structure (mucosa, submucosa, muscularis propria, and serosa) was clearly resolved in US image, and a uniformly distributed vasculature in the submucosa region was seen in PA image. H&E images revealed rich blood vessels throughout the submucosa that are well correlated with photoacoustic image findings.

The second patient (FIGS. 33E, 33F, 33G, and 33H) was a 68-year-old man with rectal cancer treated with chemotherapy and radiation. Abdominoperineal resection was performed to remove the rectum and anus. Representative T2-weighted MRI and DWI images showed a high signal intensity typical of rectal tumor, with a void region of low signal at approximately 6 to 9 o'clock, which may have indicated normal tissue with a good therapeutic response. US showed a distorted morphology in the region marked by the white box, which corresponded to the DWI-indicated hyperintensity region. The PA signal at the mucosal surface was from blood smeared along the inner wall, but no regular vasculature distribution pattern was observed inside the marked region. H&E imaging (FIG. 33H) from the interface of the normal and tumor regions demonstrated the difference in microvasculature previously described. The tumor lacked blood vessels, but normal tissue had abundant vessels marked by arrows. Pathologic evaluation revealed a 5 cm residual tumor involving the low rectum and upper portion of anus. The tumor had invaded through the muscularis propria into the perirectal tissue (pT3). FIGS. 34D, 34E, and 34F showed a normal area of the rectum imaged at the distal end with normal pathology. MRI showed no abnormal T2 signal or diffusion restriction. The US image demonstrated a normal layered structure, and the PA signal was uniformly distributed in the submucosa. The H&E image, which revealed rich blood vessels in submucosa, was well correlated with the PA image.

The third patient (FIGS. 33I, 33J, 33K, and 33L) was a 67-year-old man with two tumor masses: one in colon and one in rectum. The rectal tumor was treated with radiation and chemotherapy. The T2-weighted MRI and the DWI taken 3 months before the surgery indicated mild residual wall thickness and some post-treatment fibrosis related to the treatment. However, the previously seen rectal mass was no longer well seen on MR and DW images, indicating excellent response. Two months later, however, the surgeon discovered a viable tumor in the rectum on repeated endoscopy. The representative US image taken before rectum resection showed a large tumor centered at 11 o'clock. The majority of the malignant tissue lacked blood vessels, though a few vessels were located at the center of the tumor. The H&E image taken at approximately bulk of the tumor showed that most areas lacked blood vessels, which correlated with the PA image findings. FIGS. 34G, 34H, and 34J showed the proximal end of the rectum with normal pathology, as confirmed by the resection specimen. The US image demonstrated a normal layered structure, the PA signal was uniformly distributed in the submucosa, and the H&E image revealed rich blood vessels in submucosa.

The fourth patient (FIGS. 33M, 33N, 33O, and 33P) was a 64-year-old man treated with initial radiation and chemotherapy that resulted in a clinical complete response based on MRI, endoscopy, and clinical examination at the time of treatment completion. The patient was closely surveilled with quarterly MRIs consistently showing low T2 signal in the right rectal wall corresponding with fibrosis; endoscopic evaluations also showed no abnormality within the rectal lumen except a benign appearing scar. DWI also showed no hyperintensity to suggest a recurrent tumor; representative T2-weighted MRI and DWI images taken 9 months into his surveillance are given in FIG. 33M. However, several weeks after this normal-appearing MRI, endoscopic evaluation of the prior tumor side revealed new nodularity; biopsies taken at that time were found to contain recurrent cancer, and the patient then underwent surgical resection of the rectum. The PA image (FIG. 33O) revealed a region lacking of vasculature and co-registered US showed some acoustic attenuation contrast, as indicated by a white dashed line box. Surgical path revealed a recurrence tumor of approximately 1.2 cm with a majority of the tumor mass underneath the mucosa. This example demonstrated the superior sensitivity of PAE/US over MRI and the potential of PAE/US as an imaging tool for rectal cancer post treatment surveillance.

The PAE-CNN and US-CNN models described in Example 5 were used to evaluate the above five patients. Table 3 lists results for the five patients imaged in vivo. The expected probability of normal rectal tissue was 100% for the normal area in the group and 0% for the residual tumor area. The PAE-CNN model yielded average probabilities of 11.47%, 15.59%, 39.31% and 32.47% for the first patient's residual tumor area (1-T), second patient's residual tumor area (2-T), third patient's residual tumor area (3-T), and fourth patient's residual tumor area (4-T), respectively. However, the US-CNN model yielded probabilities of 49.38%, 48.06%, 32.39% and 63.72% accordingly, with one misclassification when 50% was used as a threshold. In normal regions, the PAE-CNN model output average probabilities for the first (1-N), second (2-N), third (3-N), fourth (4-N) and fifth patient (5-N) of 89.42%, 71.51%, 81.48%, 74.32% and 94.45%, with no misclassification. However, US-CNN provided 47.32%, 83.73%, 75.90%, 87.93% and 75.52%, with one misclassification. The numbers in the US and PA panels of FIGS. 33B, 33C, 33F, 33G, 33J, 33K, 33N, 33O, 34A, 34B, 34D, 34E, 34G, 34H, 34J, 34K, 34M and 34N are the corresponding average US-CNN and PAE-CNN outputs indicating the probability of normal.

TABLE 3
CNN Classifier tested on in vivo cases

| Lesion | | | | |
|---|---|---|---|---|
| Area | Probability of normal | | | |
| Case | Photo acoustic | Ultrasound | Expected | Pathologic result |
| 1-T | 11.47% | 49.38% | 0% | pT2 residual |
| 2-T | 15.59% | 48.06% | 0% | pT3 residual |
| 3-T | 39.31% | 32.39% | 0% | pT3 residual |
| 4-T | 32.47% | 63.72% | 0% | 1.2 cm |
| | | | | recurrence tumor |
| 5-T | N/A | N/A | N/A | N/A |
| 1-N | 89.42% | 47.32% | 100% | Normal |
| 2-N | 71.51% | 83.73% | 100% | Normal |
| 3-N | 81.48% | 75.90% | 100% | Normal |
| 4-N | 74.32% | 87.93% | 100% | Normal |
| 5-N | 94.45% | 75.52% | 100% | Normal |

Probability of normal refers to the mean probability of normal for all tested ROIs; Expected probability that responders are classified as normal is 100%, and the probability that non-responders are classified as normal is 0%.

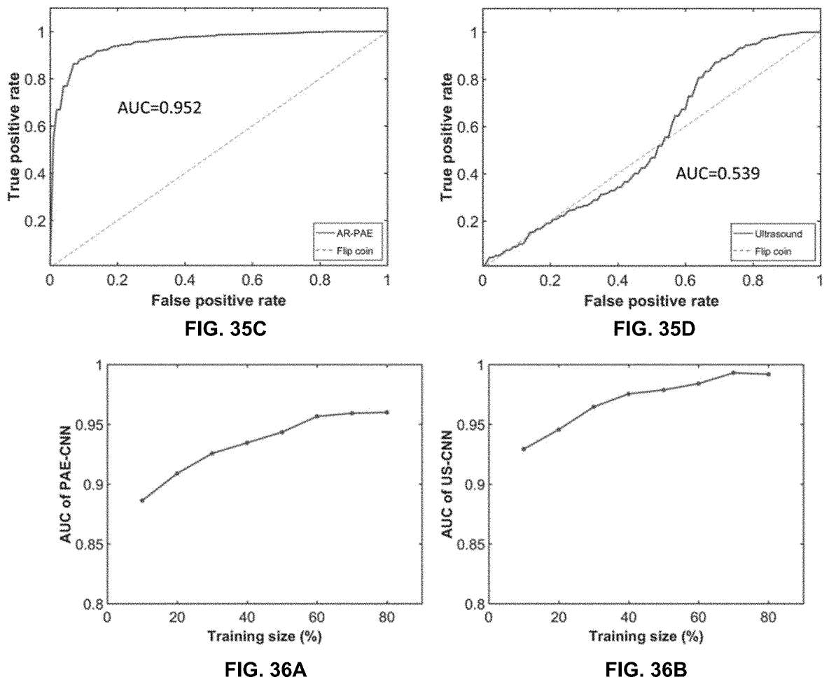

From a total number of 162 PA regions of interest (ROIs) and 162 US ROIs obtained from the five patients, ROCs were computed as shown in FIGS. 35A and 35B; the areas under ROC are 0.976 and 0.758 for PAE-CNN and US-CNN, respectively. ROCs were also computed using mixed in vivo and ex vivo data with a total number of 136 PA ROIs and 136 US ROIs obtained from four in vivo and one ex vivo non-responders, and three ex vivo responders (Table 1 and Table 3). As shown in FIGS. 35C and 35D, AUC of 0.952 and 0.539 are obtained from PAE-CNN and US-CNN models, respectively.

### Example 8: AR-PAM Flowmetry Using US-PAM Probe

To evaluate the feasibility of performing PA flowmetry of rectal tissues using the US-PAM probe described herein, the following experiments were conducted.

PA flowmetry is an emerging technique that offers significant advantages over Doppler US in measuring low flow velocities in the range of several mm/s to few tens of mm/s and is suitable for evaluating rectal tumor bed microvasculature to characterize the tumor microenvironment. To implement PAM flowmetry using imaging data obtained by the in vivo US-PAM probe described in Example 1, photoacoustic A-lines obtained by the probe were appropriately time-windowed and cross-correlated, and the displacements of peak correlations were recorded. These displacements were then converted to a velocity map in a manner analogous to range-gating in conventional pulsed wave Doppler US. The final velocity map was converted to a velocity plot using a color Doppler scheme with red to orange representing increasing positive velocity and blue to cyan representing increasing negative velocity. The velocity plot was converted to a polar display and superimposed on the US image to yield a co-registered PAM-velocity map and US.

FIG. 29A is a co-registered PAM-velocity map/US at one edge of a tumor bed along the rectal wall and FIG. 29B is a similar co-registered PAM-velocity map/US obtained from the normal rectal tissue of the same subject. The reoccurrence tumor bed had a sparse velocity profile but higher velocity and densely packed vessels around the tumor bed periphery as marked by arrows. In the normal rectal tissue (FIG. 29B), the velocity was uniformly distributed with higher and lower values that may correspond to clusters of arterioles and venules. This finding suggests that PAM-velocity parameters may be useful for assessing tumor bed blood flow and to evaluate response to treatment.

The velocity estimation will be validated with a series of phantom studies of well controlled flow velocities in different scattering backgrounds. Methods for total flow estimation using a spectral broadening method and flow estimation using speckle tracking will be investigated. These methods are less dependent on Doppler angle estimation and may be more robust. Histograms of velocity in the residual tumor bed and normal rectal tissue will be analyzed statistically to extract features representative of cancer vs. normal rectal tissue.

### Example 9: Refinement of CNN Models for Identifying Healthy and Cancerous Colon Tissues

To assess the effectiveness of training the CNN models for identifying healthy and cancerous tissues as described herein, the following experiments were conducted.

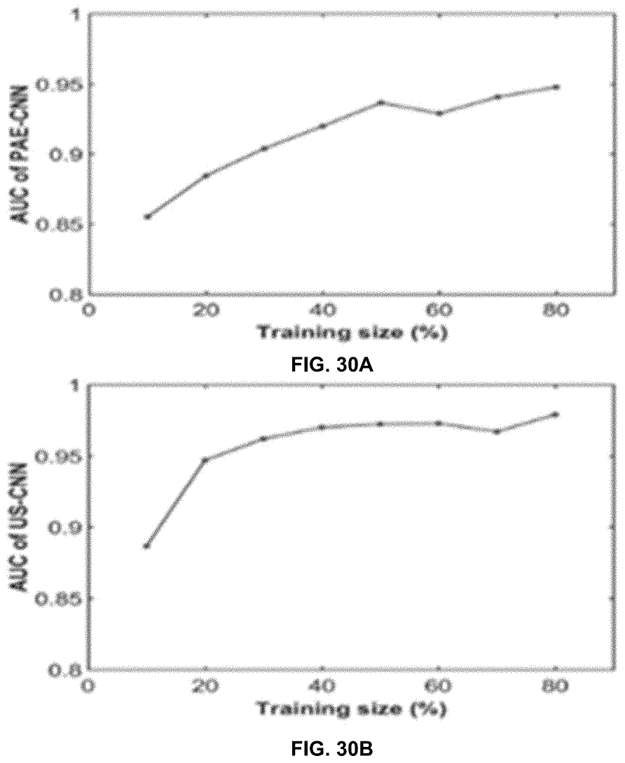

Since data to train and validate the PAM-CNN and US-CNN models are typically limited, the amount of training data sufficient to reliably train the CNN models of Example 4 was evaluated. Validation AUC for different training sizes was calculated. 20% data of the total data available for use in training and validation was used for validation, and the proportion of available data used for training was varied from 10% to 80% in steps of 10%. Validation AUC was plotted against training size for the PAE-CNN and US-CNN models as shown in FIGS. 30A and 30B, respectively. The AUC curve was almost flat for US-CNN trained using 30% or more of the available data for training (FIG. 30A), and changed little for PAM-CNN trained using about 50% or more of the available data for training (FIG. 30B). Based on the results of these experiments, 50% of the collected data was sufficient for training of the CNNs. However, more data, especially PAM data, would further improve the model performance. Additionally, more data will increase the robustness of the training by allowing the use of larger high-resolution images for training.

In other experiments, the CNN parameters of convolution kernel size, maxpooling kernel size, and the number of CNN layers vs the number of neurons in each layer will be fine-tuned. The convolution kernel size is determined by a tradeoff between memory efficiency, computation cost, and overfitting, and also depends on the complexity of the features to be learned. With more data, we will use high resolution images and increase the kernel size to let the neural network learn the layer and layer-like feature with the original information content in the data. The max pooling layer extracts the local maxima of the input within a kernel. Since neighboring pixels are correlated in an image after convolution, max pooling removes redundancy and decreases data dimensionality. When using higher resolution images, the max pooling kernel size will be increased accordingly to reduce computational cost. The number of network layers and number of neurons in each layer will be optimized. Without being limited to any particular theory, one hidden layer is sufficient to map any dimensional space to another, provided that this layer has enough neurons. To learn complex features, shallow networks need many hidden neurons, which can necessitate computing an unfeasibly large number of weights. Similar performance will be achieved by using multiple hidden layers with fewer nodes in each layer, which will be computationally efficient. Different network depths will be evaluated within a wide range, as well as different numbers of neurons in each layer, to determine which combination works best using a larger validation set.

In training, 1D ROIs from PAM and US B-scans were used as input images to CNNs. Misclassifications can occur in ROIs when SNRs are low. To reduce the occurrence of misclassifications, 2D ROIs will be used as input images to CNNs. 2D ROIs from a small number of sequential B-scans will reduce the dependence of CNNs on the SNR of individual 1D ROIs and therefore reduce misclassification.

PAM-CNNs and US-CNNs will be combined to enhance classification performance. As seen from Table 1 above, US-CNN identified normal colorectal tissue with good accuracy, but performed poorly on treated tumor beds with pCR due to treatment-induced tissue changes. Presenting both US and PAM images as a pair to train/validate a CNN will improve the performance as compared to the performance of PAM-CNN alone.

### Example 10: Comparison of CNN and GLM Models for Identifying Healthy and Cancerous Colon Tissues

To compare the effectiveness of CNN versus GLM models for identifying healthy and cancerous tissues as described herein, the following experiments were conducted.

The classification of cancerous and normal colorectal tissues within PA images obtained using the systems and methods described herein were compared to a traditional histogram-feature based model. Using 24 ex vivo and 10 in vivo data sets, the performances of the PAM-CNN and the traditional histogram-parameter-based classifiers in rectal cancer treatment evaluation were compared. Unlike CNN models, a generalized logistic regression (GLM) classifier did not require a large dataset for training and validation, however, imaging features must be extracted and evaluated on their diagnostic accuracy. Five PAM image histogram features were computed and used to train, validate and test GLM classifiers. The performances of the deep learning based CNN models were compared with the corresponding performance of GLM classifiers.

Briefly, 10 participants (mean age, 58 years; range 42-68 years; 2 women and 8 men) completed radiation and chemotherapy from September 2019 to September 2020 and were imaged with the PAM/US system prior to surgery. In the in vivo study, patients who had previously undergone preoperative treatment with radiation and chemotherapy were imaged in vivo before resection.

Colorectal specimens from another group of 24 patients who had undergone surgery were studied ex vivo (Table 4). In the ex vivo study, each specimen was evaluated within one hour of surgical resection and prior to formalin fixation.

TABLE 4
Lesion characteristics (24 ex vivo colorectal specimens and 10 patients)

| Lesion characteristics | Pathologic result |
|---|---|
| Ex vivo colorectal cancer (63 years) | Adenocarcinoma,T1-T3, n = 15 |
| Ex vivo treated rectal cancer | Residual adenocarcinoma, n = 3 |
| (63 years) | |
| Ex vivo treated rectal cancer | pCR, n = 3 |
| (52 years) | |
| Ex vivo normal colorectal tissue | 18 normal areas from cancer |
| | patients and five patients with |
| | only normal colorectal |
| | tissue available |
| In vivo treated rectal cancer | Recurrence tumor, residual tumor, |
| (61 years) | n = 6 |
| In vivo treated rectal cancer | pCR, n = 1 |
| (53 years) | |
| In vivo normal colorectal tissue | 10 normal areas from 7 cancer |
| | patients and three patients with |
| | only normal rectal tissue available |

Ages shown are average ages for each group.

### Example 11: Evaluation of Fiber Tip Diffuser for US-PAM Probe

To assess evaluate the performance of a fiber tip diffuser configured to diffuse the laser illumination delivered to tissues by the US-PAM probes described herein, the following experiments were conducted.

The measured output patterns of a fiber tip without a diffuser (normal) and a 20:10 fiber tip diffuser are shown in FIG. 1B. The output patterns were obtained using matched input laser energy. As illustrated in FIG. 1B, a larger spot was observed with the diffuser tip, and the laser intensity distribution was more homogenized than normal fiber tip output.

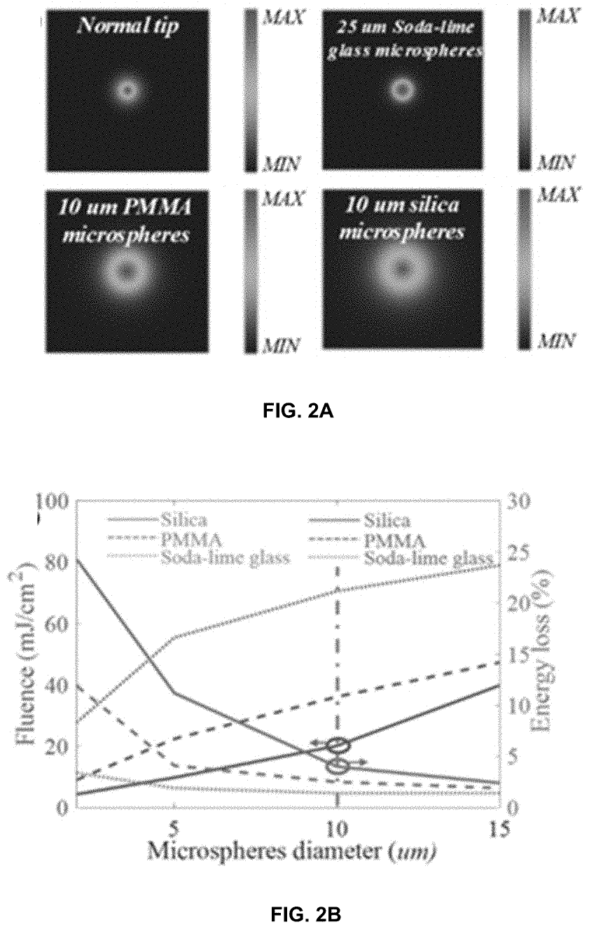

To evaluate fiber tip diffuser parameters and scattering effect, simulations were performed to examine the influence of microsphere size and material refractive index on the tip scattering effect, as shown in FIG. 2A and FIG. 2B. Zemax's Mie scattering model was used to simulate the output laser patterns in water medium of different diffuser tips made of different microspheres manufactured on 1 mm core diameter multimode fibers (0.5 N.A., Thorlabs), as shown in FIG. 2A. Commonly used microsphere materials, such as soda-lime glass (n=1.52), methyl methacrylate (PMMA) (n=1.49), and silica (n=1.458) microspheres with the maximum 20:10 diffuser concentration, were used for the simulation summarized in FIG. 2A.

Without being limited to any particular theory, different microsphere materials and different microsphere sizes have distinctive scattering effects. FIG. 2B summarizes the fluence (blue lines) and energy loss (red lines) of different microsphere materials with respect to changes of microsphere diameter in a water medium. As illustrated in FIG. 2B, silica microspheres exhibited the best scattering effects but also had the highest energy loss among all the simulated materials. By using 10-μm diameter silica microspheres (marked by dotted vertical line), a reasonable scattering effect of 6.5 times fluence reduction with acceptable laser energy loss (<5%) was realized. Larger microsphere diameters had reduced scattering effects, while smaller microsphere diameters produced much higher energy loss due to a large scattering angle and back scattering.

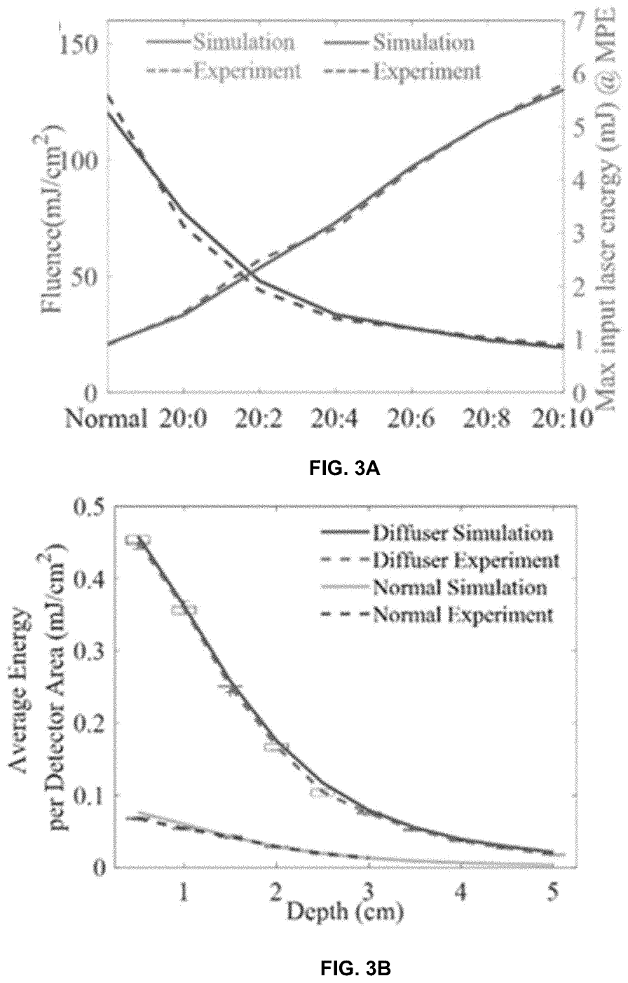

Without being limited to any particular theory, the concentration of silica microspheres is another parameter influencing the performance of the fiber tip diffusers disclosed herein, as illustrated in FIG. 3A for both simulation and experimental results in water. Fluence (blue lines) and maximum input laser energy @ MPE (red lines) were plotted as a function of diffuser concentration in FIG. 3A. At the same input energy level (i.e. 4 mJ/pulse in simulation and experiment), the output fluence dropped from around 130 mJ/cm² for a normal fiber tip to 20 mJ/cm² for a 20:10 fiber tip diffuser. With the increase of silica microsphere concentration, the maximum input laser energy without exceeding tissue surface MPE also increased, from 0.9 mJ/pulse with the normal fiber tip to 5.7 mJ/pulse with a 20:10 fiber tip diffuser. This input energy increase induced large photoacoustic signal enhancement without exceeding the surface MPE due to the diffused laser spot, as described in additional detail below.

In order to analyze the fiber tip diffuser's scattering effect inside a tissue, a calibrated 0.4% intralipid solution of 4 cm⁻¹ reduced scattering coefficient (μs′) and 0.02 cm⁻¹ absorption coefficient (pa) was used for both simulated and experimental measurements. As observed in the experimental measurements, the light is scattered quickly in the intralipid solution, and existing energy detectors were unable to measure over the relatively large area illuminated in the intralipid solution by the various optic fiber designs for the fluence measurement. Consequently, average energy, defined as the total detected energy averaged by the entire energy detector sensing area, was used to assess fluence within the intralipid solution. Based on this definition, a higher average energy corresponded to a higher fluence around the energy detector sensing area. To obtain simulation results, the Henyey-Greenstein scattering model was applied for simulating biological tissue, and a 25 mm diameter detector area was used to simulate the 25 mm diameter energy detector (Coherent Inc., J-25 MB-HE) used in the experimental measurements.

Both simulated and experimental energy distributions of different depths in 0.4% intralipid solution are shown in FIG. 3B. The performance of a single 20:10 fiber tip diffuser and a normal fiber tip was compared by measuring the fluence propagating through 5-50 mm thickness of the calibrated intralipid solution. During the simulation and experiment, both 20:10 fiber tip diffuser and normal fiber tips are controlled at MPE input energy at tissue surface. Both 20:10 fiber tip diffuser and normal fiber tip demonstrated good agreements between simulation and experiment, where fluence decreased with the increase of depth due to scattering-induced laser spot expansion. With reduced laser energy input, which is limited by MPE, the normal fiber that delivered 0.9 mJ/pulse showed much weaker average energy in all the depths, nearly ⅙ of that of the fiber with a 20:10 diffuser that delivered 5.7 mJ/pulse.

Without being limited to any particular theory, according to the well-established relationship between photoacoustic signal and laser fluence, the initial pressure generated by photoacoustic effect is characterized as having a linear relationship with laser fluence and tissue absorption. With a high fluence distribution inside the tissue, a high photoacoustic signal should be generated under the same tissue absorption parameter. Thus, a diffuser with a higher energy input elicits an enhanced photoacoustic signal as compared to a normal fiber with limited input energy.

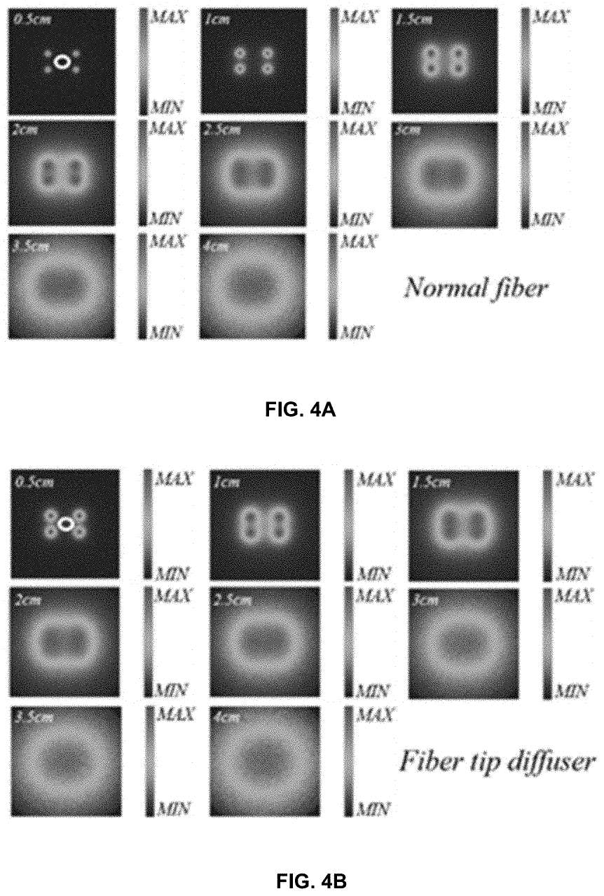

Applying the disclosed fiber tip diffuser to a transvaginal probe, the imaging quality with simulated biological tissue was also assessed. Fluence distribution under different depths was simulated using a 4-fiber transvaginal probe configuration. The photoacoustic system setup consisted of a fully programmable clinical US system (EC-12R, Alpinion Medical Systems, Republic of Korea), and a Nd:YAG laser (Symphotics-TII, LS-2134, Camarillo, Calif.) pumping a pulsed, tunable (690-900 nm) Ti-sapphire laser (Symphotics TII, LS-2122). The transvaginal US/PAT probe consisted of a 128-channel endo-cavity US transducer (EC3-10, Alpinion Medical Systems, Republic of Korea) with a 6 MHz central frequency, 80% bandwidth, elevation height of 6 mm, and 145.5-degree field of view, surrounded by four 1 mm core diameter multimode fibers (0.5 N.A., Thorlabs) for light delivery. FIGS. 4A and 4B show the simulated laser fluence distributions at different depths (5-40 mm) within the calibrated intralipid solution using a normal fiber ending (FIG. 4A) and the 20:10 fiber tip diffuser (FIG. 4B), respectively. Comparing FIG. 4A with FIG. 4B, the four laser spots merged faster using the 20:10 diffuser relative to the normal fiber.

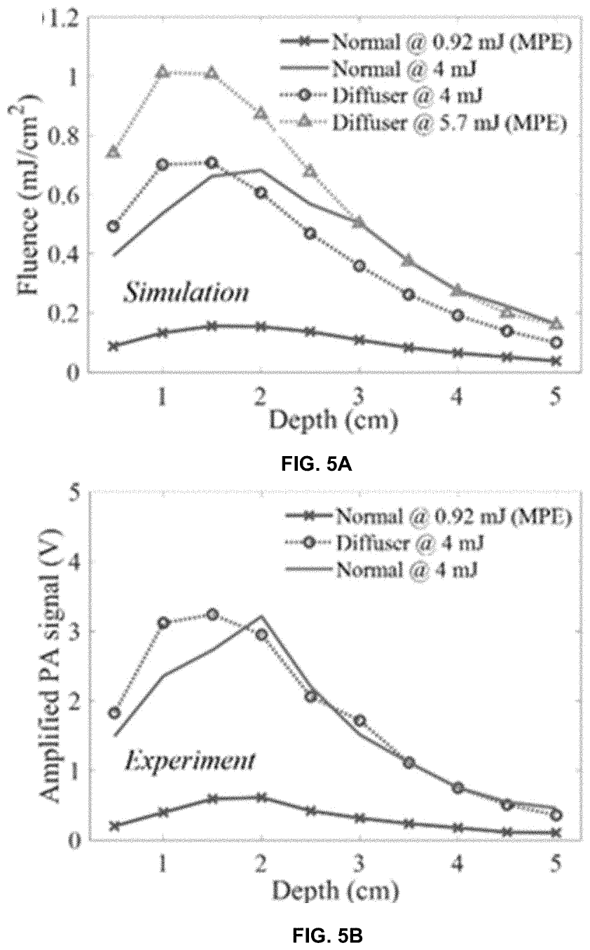

Simulated fluence depth information at the center region of the 4 optic fibers representative of a transvaginal probe, where the imaging target is located, represented as superimposed white circular areas in FIGS. 4A and 4B, inside calibrated intralipid solution with different diffuser concentrations and different input energies are summarized in FIG. 5A. Based on the simulation results shown in FIG. 5A, the fluence distribution in calibrated intralipid solution initially increased at around 1.5 cm depth, reached a peak fluence, and fluence decreased as the depth continued to decrease. For the 20:10 diffuser tip design, the peak fluence occurred at about 1.5 cm-2 cm. At 1 cm depth, 15 dB fluence enhancement was observed with 20:10 diffuser (green line, 5.7 mJ/pulse) relative to the fluence produced by the normal fiber (blue line, 0.9 mJ/pulse). With matched laser energy inputs (i.e. 4 mJ/pulse), no differences in the fluence distributions produced by diffuser tips with different diffuser concentrations were observed (see purple and orange lines of FIG. 5A).

Experimental measurements were conducted to validate the simulated results described above. Amplified photoacoustic signals elicited by laser pulses delivered through various optic fiber end configurations were recorded using a commercial ultrasound transducer (EC3-10). The amplified photoacoustic signals from the commercial ultrasound transducer are summarized in FIG. 5B. The trends of photoacoustic signal strengths shown in FIG. 5B were similar to the fluence trends illustrated in FIG. 5A, described above. Similar trends are shown in the experiments (FIG. 5B) as the simulation (FIG. 5A) in that the optic fiber end configuration that included the 20:10 diffuser concentration enabled more energy to be injected into the tissue without exceeding tissue surface MPE, thus generating higher photoacoustic signals. In the experimental results summarized in FIG. 5B, data for the 4 fiber diffuser tips reaching MPE energy input levels (green line in FIG. 5A) were not obtained due to the total energy limitation of the Ti:sapphire laser used in these experiments, which was capable of producing only up to 4.5 mJ/pulse per fiber for the 4 fibers. Any discrepancy between experiment and simulation data summarized in FIGS. 5A and 5B was likely due to system alignment in the experimental set-up.

PAM and US images were obtained using the probe described above and illustrated in FIGS. 10A, 10B, and 10C. For training and validation, three to five regions of interest (ROIs) were selected at uniformly spaced locations on each PAM or US B-scan image acquired from normal regions or a tumor bed (FIGS. 12A and 12B. The red ring in FIGS. 12A and 12B are indicative of mucosa vasculature, which is continuous in the normal image of FIG. 12B. The blue rectangles denoted selected ROIs are uniformly spaced along the perimeter of each image. In the cancer image (FIG. 12A), the dark zones and discontinuities in the red ring from approximately 9:00 to 1:00 o'clock indicate the presence of a tumor, so the ROIs are uniformly spaced within that segment. A total of 2600 US ROIs (1262 normal and 1496 cancerous) and 2004 PA ROIs (1207 normal and 797 cancerous) were compiled from 24 patients' ex vivo images and 10 patients' in vivo images (Table 4). Two ex vivo samples that showed a low signal-to-noise ratio (SNR) on PAM images due to a laser energy problem were excluded in training PAM-CNN and PAM-GLM models. For the US-CNN and US-GLM models, all 24 ex vivo and all 10 in vivo patient data were used.

The total of 2004 PA ROIs and a total of 2600 US ROIs were divided into two discrete data sets for model training/validation and for testing, respectively. The training set included all ex vivo cases (see Table 4) and half of the in vivo patient data. Of the training set ROIs, 80% were used for training with the remainder for internal validation. The testing set contained the other half of the in vivo patient data.

We used selected image features of ROIs to develop PAM-GLM and US-GLM models. To calculate the histogram of each ROI, we divided the ROI into 32 bins. The bar height of each bin was then computed by dividing the number of pixels with a given value in an associated range by the size of the image. From the histogram of each ROI, we then extracted five features: mean, standard deviation, skewness, kurtosis, and energy.

All the PAM and US features showed significant differences between malignant and normal colorectal tissues (p<0.05) (Appendix FIG. 1S and FIG. 2S). Therefore, all these features were considered as potential candidates when building PAM-GLM and US-GLM models. To prevent model overfitting, the Spearman's correlation coefficient between each of the histogram features was calculated (Appendix, Table 1S). We developed PAM-GLM classifiers using each histogram feature separately, as well as using combinations of features with low correlation values. The mean AUCs of the training/validation and testing data sets as well as their 95% confidence of interval were computed for each classifier. The same process was followed to construct US-GLM classifiers.

To remove bias in selecting in vivo data for training and validation, we trained the classifiers 10 times. The training/validation and testing data sets are the same as those used for CNN models described below.

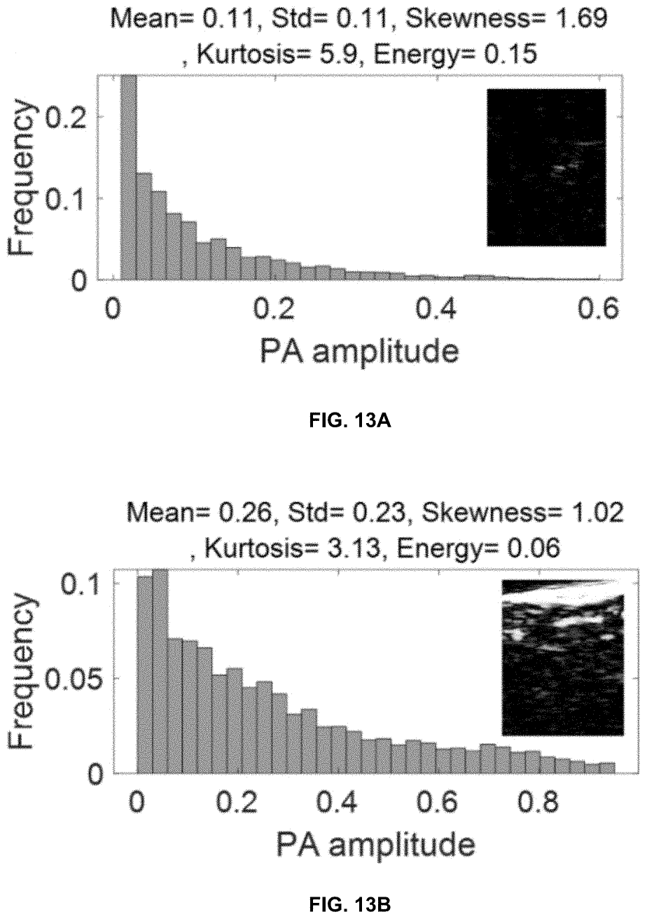

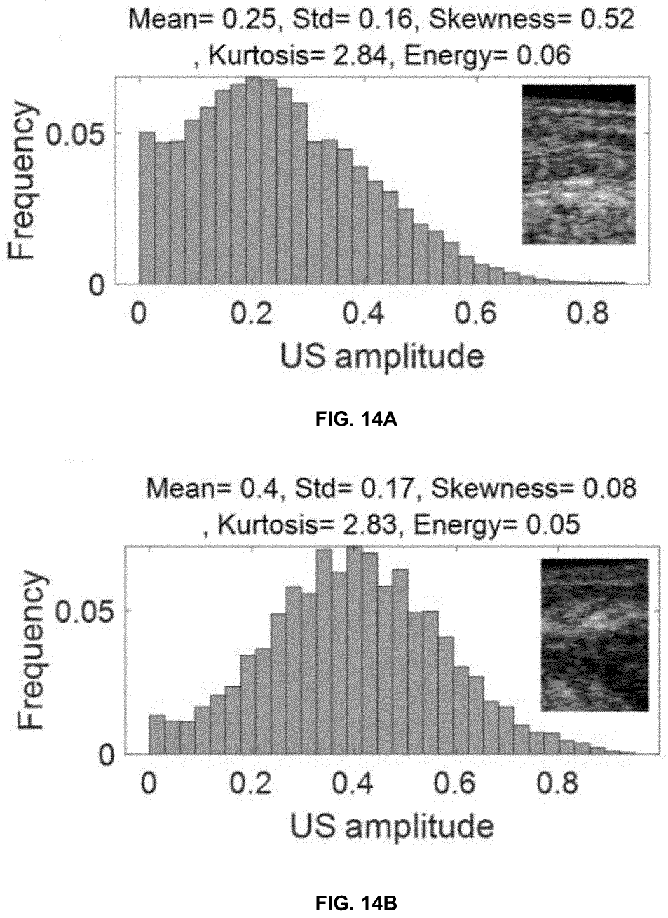

FIGS. 13A and 13B (PAM-GLM) and FIGS. 14A and 14B (US-GLM) show examples of the first order statistical features calculated from malignant rectal tissue ROIs (shown in FIG. 12A) and normal rectal tissue ROIs (shown in FIG. 12B). As shown in FIGS. 13A and 13B, in PAM ROIs, the malignant tissue has a lower mean and standard deviation, while the other three features are higher. In FIGS. 14A and 14B, malignant US ROIs show a lower mean and standard deviation than that of the normal US ROIs.

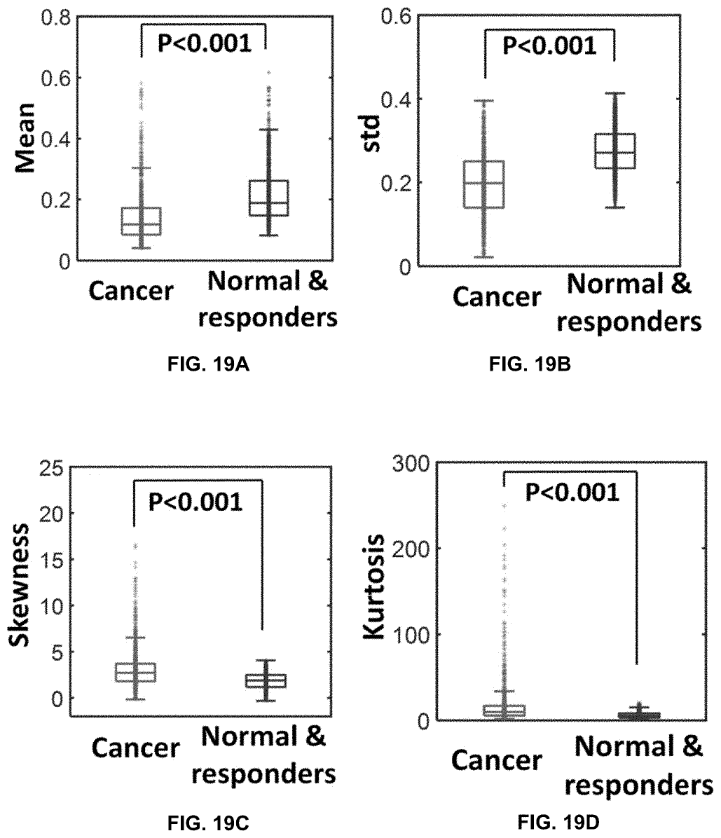

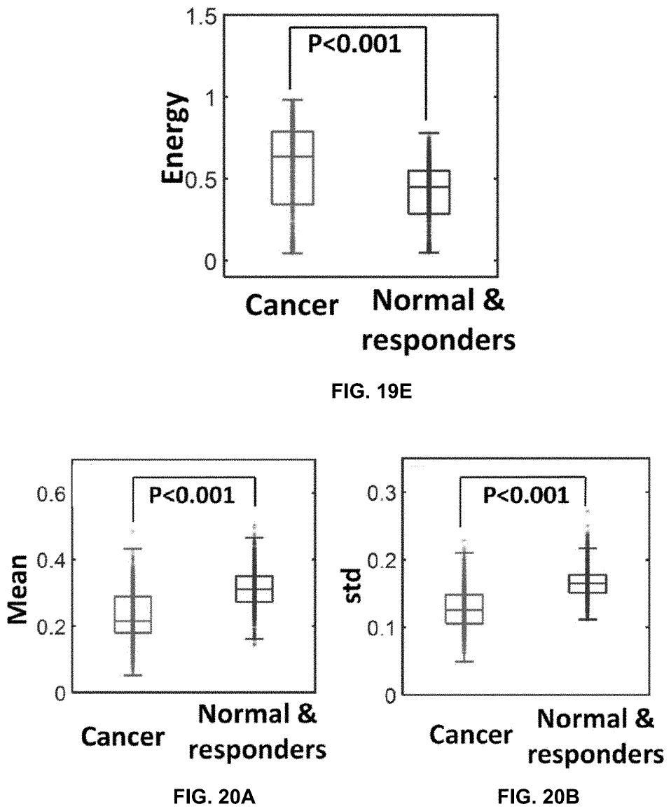

FIGS. 19A, 19B, 19C, 19D, 19E, and 19F show the boxplots of the histogram features of the PAM ROIs. The p-value for each feature, calculated from a two-sided statistical t-test, is indicated on each plot. All features are statistically significant (p<0.05), however, they are not equally important. To assess the importance of each feature a regression model was fit to each feature separately, using all the available data (ex vivo and in vivo patients), and then the AUC of the fitted model was determined. As shown in Table 5 below, Std, Mean, and Kurtosis respectively provide the highest AUC values among all the features of PAM images. While Std and Mean are highly correlated, the correlation value between Mean and Kurtosis is less than 0.5 (Table 6). Therefore, these two features are used together to develop PAM-GLM classifiers.

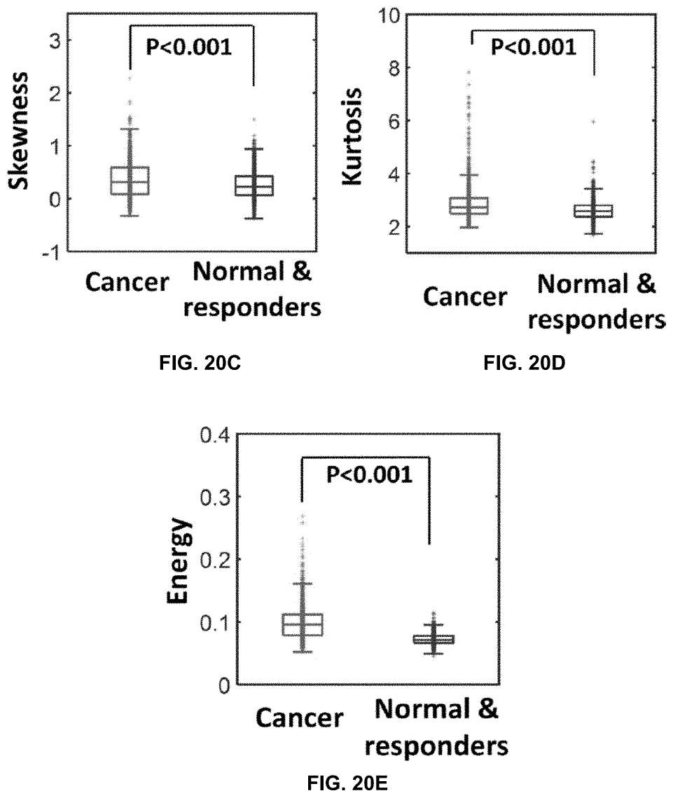

Similarly, boxplots of the five features from US ROIs are given in FIGS. 20A, 20B, 20C, 20D, 20E, and 20F, and the AUC feature values of the fitted model are shown in Table 5. Based on this table, Std, Energy, and Mean are respectively the most important features of the US images. However, they all are highly correlated with each other (Table 7).

TABLE 5
AUCs of the fitted regression model developed using
features of PAM and US images

| Feature | AUC (PAM) | AUC (US) |
|---|---|---|
| Mean | 0.76 | 0.81 |
| Std | 0.79 | 0.86 |
| Skewness | 0.71 | 0.57 |
| Kurtosis | 0.73 | 0.62 |
| Energy | 0.70 | 0.85 |

TABLE 6
Spearman's correlation between histogram
features of the PAM images

| | Mean | Std | Skewness | Kurtosis | Energy |
|---|---|---|---|---|---|
| Mean | 1 | 0.79 | 0.72 | 0.42 | 0.79 |
| Std | — | 1 | 0.75 | 0.57 | 0.57 |
| Skewness | — | 0.57 | 1 | 0.9 | 0.77 |
| Kurtosis | — | — | — | 1 | 0.5 |
| Energy | — | — | — | — | 1 |

TABLE 7
Spearman's correlation between histogram
features of the US images

| | Mean | Std | Skewness | Kurtosis | Energy |
|---|---|---|---|---|---|
| Mean | 1 | 0.7 | 0.63 | 0.38 | 0.78 |
| Std | — | 1 | 0.15 | 0.35 | 0.9 |
| Skewness | — | 0.57 | 1 | 0.69 | 0.41 |
| Kurtosis | — | — | — | 1 | 0.41 |
| Energy | — | — | — | — | 1 |

The PAM-CNN (or US-CNN) architecture (FIG. 11) contained two sequential feature extraction layers and two fully connected layers. Briefly, each extraction layer had a convolutional layer followed by a pooling layer. Each convolutional layer uses a 3×3 kernel, and each pooling layer has a 4×4 kernel with max-pooling. These kernel sizes were selected based on the optimal validation results. The first fully connected layer was a 512-node hidden layer, and the second fully connected layer (output layer) generated two output classifications—normal or cancerous. “Normal” described a layer-like vascular distribution in a PAM image or a layer structure in a US image, and “cancerous” described an absence of the normal vasculature pattern in PAM images, or an absence of the layer structure in US images. A “softmax” activation function in the output layer generated the probabilities of each of the two possible classifications (cancer or normal) for an input image; for each input ROI of a PAM or US image, the CNN model outputted the probability of a normal classification compared to the threshold (e.g. >50% is normal). In all the other layers, a “ReLU” activation function immediately sets all negative values to zero. The ReLU activation function with a gradient of 1 for positive inputs and 0 for negative inputs ensures no exploding or vanishing gradient problem occurs.

To avoid biased selection, we trained and validated 10 PAM-CNN and US-CNN models each using all the ex vivo data and a randomly selected half of the in vivo patient data, while reserving the other half for testing. The maximum number of epochs was 20, with early stopping (a tolerance of 2 epochs) monitored by validation accuracy. If there was no increase in validation for two successive epochs, training was stopped. Stochastic gradient descent was used with a batch size of 20, and the RMSprop optimizer function was used to optimize the neural net weights. The learning rate was set to 10⁻³ with a decay of 10⁻⁵. In each model, 80% of the ROIs from the training & validation set were used to train the model, the remaining 20% were used for validation, and 20× cross validation was performed.

The ROIs of each in vivo normal or tumor bed patient images were either all used in training or all used in testing. Each of the 10 CNN models was tested on a randomly selected half of the in vivo data and generated an ROC. The overall performance of the classifier was measured by the mean AUC of the 10 models.

To obtain AUCs, the ex vivo data set was fixed for training and validation, but the five in vivo data set for training and validation and the five in vivo data set for testing were interchanged randomly for 10 times, and the 10 AUCs was used to generate the mean value of AUC.

Table 8 shows the mean AUCs and 95% confidence of interval for PAM-GLM classifiers developed using single features, as well as feature pairs that are weakly correlated (based on Table 6). As can be seen, the “Mean-Kurtosis” combination results in a better testing performance than “Mean” alone, and a better training performance than “Kurtosis” alone. In the case of US-GLM (Table 9), the classifier which is built using “Std” alone performs best on both training and testing data sets (mean AUCs of 0.86 and 0.66 for training and testing data sets, respectively).

TABLE 8
Training and testing mean AUC values for PAM-GLM
classifiers developed using different combinations of
weakly correlated features. The 95% confidence of interval
values are also shown in front of each mean AUC value.

| Feature | Training AUC | Testing AUC |
|---|---|---|
| combinations | (95% CI) | (95% CI) |
| Mean | 0.77 (0.767-0.777) | 0.80 (0.793-0.807) |
| Std | 0.79 (0.788-0.793) | 0.76 (0.746-0.770) |
| Skewness | 0.71 (0.708-0.719) | 0.82 (0.815-0.825) |
| Kurtosis | 0.73 (0.724-0.734) | 0.82 (0.817-0.827) |
| Energy | 0.72 (0.712-0.727) | 0.74 (0.724-0.758) |
| Mean, Kurtosis | 0.74 (0.732-0.743) | 0.82 (0.808-0.820) |
| Std, Energy | 0.80 (0.799-0.807) | 0.76 (0.750-0.773) |
| Kurtosis, Energy | 0.75 (0.744-0.750) | 0.81 (0.805-0.817) |

TABLE 9
Training and testing AUC values for US-GLM classifiers
developed using different combinations of weakly correlated
features. The 95% confidence of interval values are also
shown in front of each mean AUC value.

| Feature | Training AUC | Testing AUC |
|---|---|---|
| combinations | (95% CI) | (95% CI) |
| Mean | 0.82 (0.818-0.820) | 0.64 (0.629-0.657) |
| Std | 0.86 (0.860-0.862) | 0.66 (0.650-0.674) |
| Skewness | 0.59 (0.587-0.591) | 0.42 (0.405-0.443) |
| Kurtosis | 0.64 (0.635-0.639) | 0.34 (0.326-0.344) |
| Energy | 0.85 (0.851-0.854) | 0.61 (0.600-0.621) |
| Mean, Kurtosis | 0.82 (0.819-0.822) | 0.60 (0.581-0.618) |
| Std, Skewness | 0.86 (0.860-0.862) | 0.65 (0.643-0.664) |
| Std, Kurtosis | 0.86 (0.858-0.860) | 0.65 (0.642-0.666) |
| Kurtosis, Energy | 0.86 (0.856-0.858) | 0.63 (0.617-0.638) |

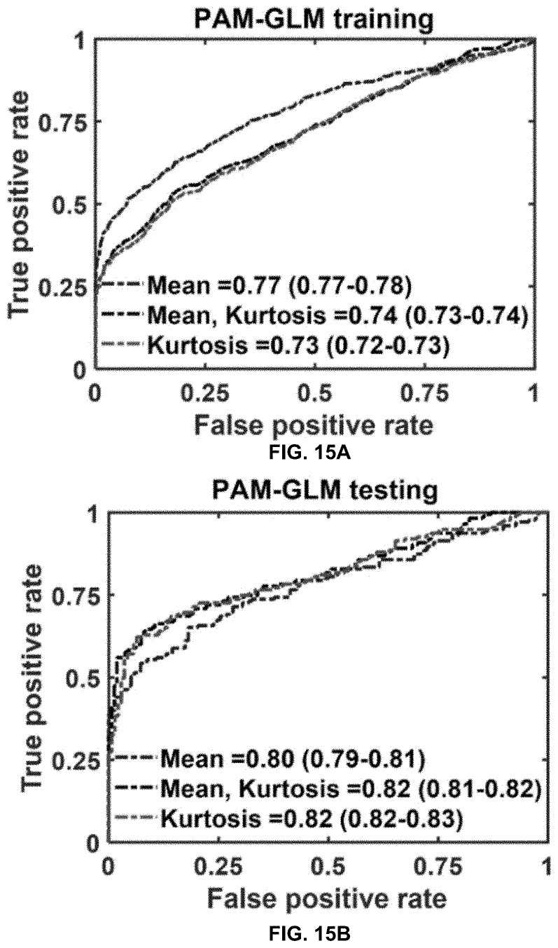

FIGS. 15A and 15B respectively show the mean training and testing ROCs of three of the best performing (based on both training and testing AUCs) classifiers developed using PAM histogram features. As shown in these plots, “Kurtosis” alone results in a slightly better performance on the testing data set than the other feature combinations (see the 95% CI values in the table). It is worth noting that although adding “Mean” to the features set negligibly lowers the AUC of the testing data set, it increases the AUC of the training data set by 0.01. Finally, the reason for the slightly poor training performance than testing for different combinations of features is that the training data set includes both in vivo and ex vivo ROIs while the testing data set contains only in vivo ROIs. Overall, our in vivo data have demonstrated slightly better classification between malignant and normal colorectal tissue than the ex vivo data.

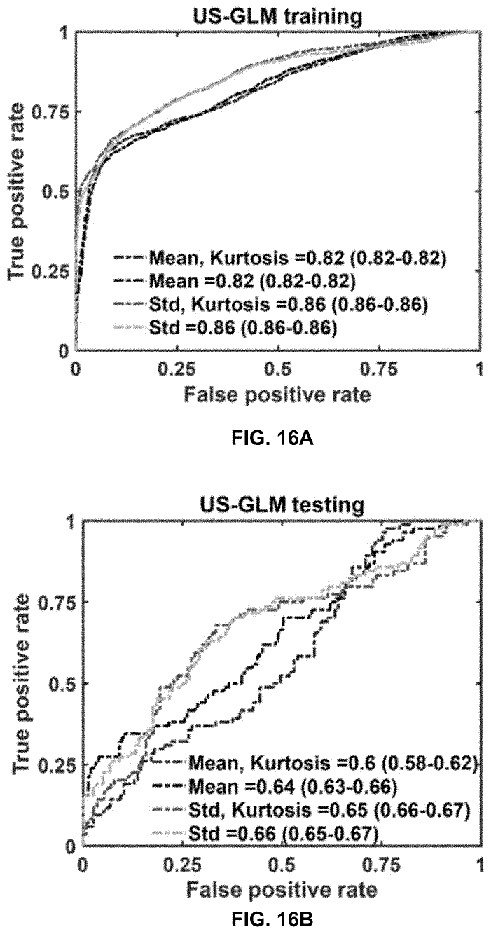

In the case of US-GLM, using the “Std” histogram feature demonstrates the best prediction AUC of 0.68, as seen in FIG. 16B. Adding any other uncorrelated features does not improve the AUCs of the training or testing data sets as shown in FIGS. 16A and 16C.

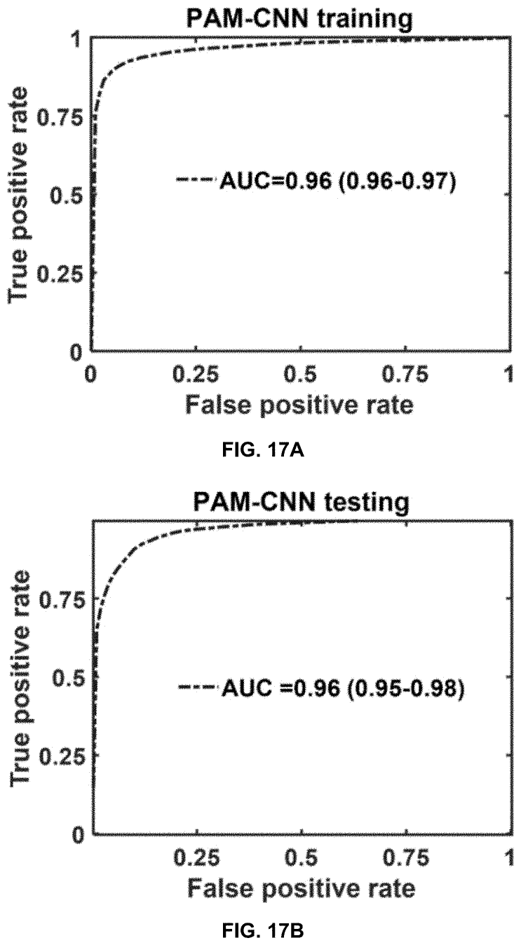

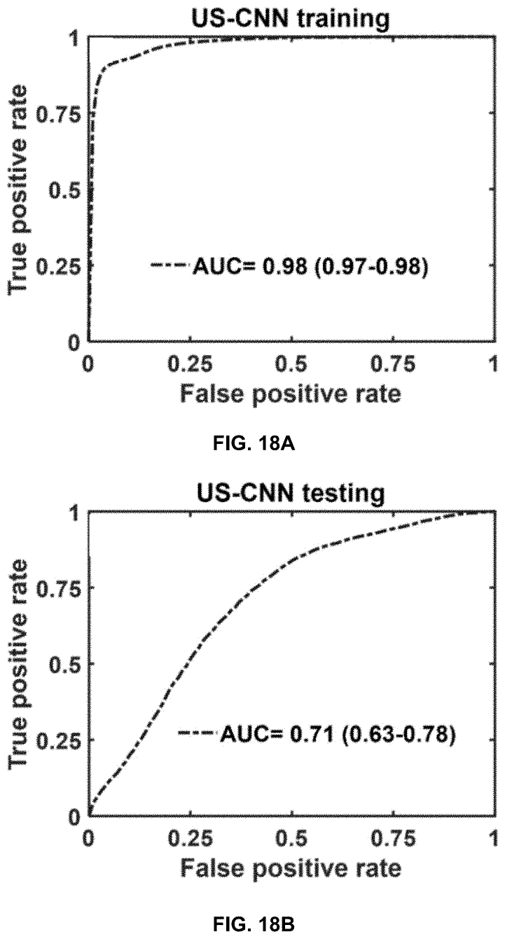

The mean ROC and AUC of the CNN models were computed from 10 CNN models, using the same shuffle method as in GLM. PAM-CNN demonstrated high performance in training and testing, with a 0.96 AUC for both (FIGS. 17A and 17B). For US-CNN (FIGS. 18A and 18B), the average AUC was 0.71 in testing.

The general architecture of the normal colon and rectal tissue consists of the mucosa (a thin layer of epithelial cells, a layer of connective tissue, a thin layer of muscle), submucosa (mucous glands, blood vessels, lymph vessels), muscularis propria (a thick layer of muscle), and serosa (an outer layer of the colon). In malignancy, the individual cell types are similar, but the architecture is distorted because cancerous cells of mucosal origin penetrate into the deeper layers of the organ. As these cells invade, the organized structure and vascular network are lost. We have observed uniform, layer-like vasculature with intense photoacoustic signals within normal rectal submucosa and in the tumor beds where complete tumor destruction has occurred. In contrast, heterogeneous and often microvascular-deficient regions have been found consistently in tumor beds with residual cancer at treatment completion [13-14]. The return of a “normal” vascular pattern to the tumor bed appears to signal complete tumor destruction, though this mechanism is not well-understood. As demonstrated, PAM-CNN captures this unique pattern and predicts pCR with a high diagnostic accuracy. PAM-GLM uses first order statistical features extracted from PAM histograms and these features do not contain spatial micro-features that can be learned by deep-learning neural networks. Thus, the performance of PAM-GLM is significantly poorer than PAM-CNNs.

The results of these experiments demonstrated that the performance of deep-learning based PAM-CNN models was significantly better than that of the PAM-GLM classifier with AUC of 0.96 (95% CI: 0.95-0.98) vs. 0.82 (95% CI: 0.81-0.83) using PAM Kurtosis. Both ultrasound-derived models (US-CNN and US-GLM) performed poorly with AUCs of 0.71 (95% CI: 0.63-0.78) and 0.66 (95% CI: 0.65-0.67), respectively. While easier to train and validate and requiring smaller data sets, GLM diagnostic performance is inferior to CNN models.

What is claimed is:

1. A system for determining a probability of normal rectal tissue composition within a region of interest of an ultrasound or photoacoustic image of the rectal tissue, the system comprising a computing device with at least one processor and a non-volatile computer-readable memory, the non-volatile computer-readable memory containing a plurality of instructions executable on the at least one processor, the plurality of instructions comprising a CNN component configured to:
receive at least one of a photoacoustic image and an ultrasound image;
select a region of interest within the at least one of a photoacoustic image and an ultrasound image;
transform the region of interest into the probability of normal rectal tissue composition using a CNN model; and
display the probability of normal rectal tissue composition to an operator of the system.

2. The system of claim 1, wherein the CNN model comprises a first and second sequential feature extraction layers, each feature extraction layer comprising a convolutional layer followed by a pooling layer, two fully connected layers connected to the second feature extraction layer.

3. The system of claim 2, wherein each convolutional layer uses a 3×3 kernel, and each pooling layer has a 2×2 kernel with max-pooling.

4. The system of claim 3, wherein the two fully connected layers comprise a hidden layer with 512 nodes connected to the pooling layer of the second feature extraction layer.

5. The system of claim 4, wherein the two fully connected layers further comprise an output layer with 2 nodes connected to the hidden layer.

6. The system of claim 5, wherein the output layer comprises a ‘softmax’ activation function configured to predict the probability of a classification of the at least one of a photoacoustic image and an ultrasound image, the classification comprising one of normal tissue or cancerous tissue.

7. The system of claim 6, wherein the CNN model is configured to transform the region of interest of the photoacoustic image into the probability of normal rectal tissue composition.

8. The system of claim 7, wherein the CNN model is configured to transform the region of interest of the ultrasound PA image into the probability of normal rectal tissue composition.

9. The system of claim 1, further comprising:
an endorectal imaging probe for obtaining co-registered ultrasound and photoacoustic images, the probe comprising:
a toroidal ultrasonic transducer mounted to an outer surface of the imaging head to detect acoustic signals produced outside of the imaging head, the toroidal ultrasonic transducer comprising a center hole aligned perpendicularly to the longitudinal axis of the probe, the toroidal ultrasonic transducer operatively connected to a remote pulser/receiver device via an ultrasonic transducer cable extending distally through the hollow axle;
an optical fiber coupled to a light source at a proximal end and extending distally through the hollow axle to a distal fiber end positioned within the imaging head; and
a prism positioned within the imaging head to direct light delivered through the optic fiber to a segment of multimode optical fiber positioned within the center hole of the transducer, the segment of multimode optical fiber configured to direct light perpendicularly outward from the imaging head.

10. An endorectal imaging probe for obtaining co-registered ultrasound and photoacoustic images of a rectal tissue of a subject, the probe comprising:
a handle comprising an integrated stepper motor and a light source;
a hollow shaft containing a hollow axle, the hollow axle coupled to the stepper motor at a proximal end;
an imaging head coupled to a distal end of the hollow axle, the imaging head comprising:
a toroidal ultrasonic transducer mounted to an outer surface of the imaging head to detect acoustic signals produced outside of the imaging head, the toroidal ultrasonic transducer comprising a center hole aligned perpendicularly to the longitudinal axis of the probe, the toroidal ultrasonic transducer operatively connected to a remote pulser/receiver device via an ultrasonic transducer cable extending distally through the hollow axle;
an optical fiber coupled to a light source at a proximal end and extending distally through the hollow axle to a distal fiber end positioned within the imaging head; and
a prism positioned within the imaging head to direct light delivered through the optic fiber to a segment of multimode optical fiber positioned within the center hole of the transducer, the segment of multimode optical fiber configured to direct light perpendicularly outward from the imaging head.

11. The probe of claim 10, wherein the segment of multimode optical fiber comprises a fiber tip diffuser at an end opposite to the prism.

12. The probe of claim 10, further comprising a water channel positioned within the handle and a water balloon positioned over the imaging head, the water channel configured to transfer water into the water balloon to enhance acoustic coupling of the imaging head with the rectal tissue.

13. A computer-implemented method for determining a probability of normal rectal tissue composition within a region of interest of an ultrasound or photoacoustic image of the rectal tissue, the method comprising:
receiving, using the computing device, at least one of a photoacoustic image and an ultrasound image;
selecting, using the computing device, a region of interest within the at least one of a photoacoustic image and an ultrasound image;
transforming, using the computing device, the region of interest into the probability of normal rectal tissue composition using a CNN model; and
displaying, using the computing device, the probability of normal rectal tissue composition to an operator of the system.
